SEQUENCE LISTING (1 sequence)

SEQ ID NO: 1 - protein, 16 aa, Homo sapiens
APHNDSATGEPEASSG

(19) United States
(12) Patent Application Publication
(10) Pub. No.: US 2011/0183948 A1
(43) Pub. Date: Jul. 28, 2011

(54) TREATMENT OF FIBROTIC CONDITIONS USING HEDGEHOG INHIBITORS

(75) Inventors: Arnold J. Levine, Doylestown, PA (US); Elizabeth Guilfoyle Trehu, Duxbury, MA (US)

(73) Assignee: INFINITY PHARMACEUTICALS, INC., Cambridge, MA (US)

(21) Appl. No.: 13/007,423

(22) Filed: Jan. 14, 2011

Related U.S. Application Data
(60) Provisional application No. 61/295,372, filed on Jan. 15, 2010.
(60) Provisional application No. 61/409,888, filed on Nov. 3, 2010.

(51) Int. Cl. A61K 31/4355; A61K 31/56; A61K 31/573; A61K 31/58; C07D 491/107; A61P 11/00; A61P 1/16; A61P 9/00; A61P 17/00; A61P 1/00; A61P 13/12; A61P 25/00; A61P 7/00; A61P 9/10; A61P 35/02; A61P 35/00; C12N 5/071

(52) U.S. Cl. 514/171; 514/278; 546/15; 435/325

(57) ABSTRACT

Provided is a method of treating a fibrotic condition with a hedgehog inhibitor.

### CROSS REFERENCE TO RELATED APPLICATIONS

This application claims the benefit of priority to U.S. Provisional Application Ser. No. 61/295,372, filed Jan. 15, 2010; and U.S. Provisional Application Ser. No. 61/409,888, filed Nov. 3, 2010. The contents of all of the aforesaid applications are hereby incorporated by reference in their entirety. A PCT patent application entitled “Treatment of Fibrotic Conditions using Hedgehog Inhibitors,” filed Jan. 14, 2011 with the U.S. Receiving Office and designating attorney docket number I2041-7016WO is also incorporated by reference.

### BACKGROUND

In the tissue remodeling response, a balance is struck between closing the wound rapidly and repairing the surface. The remodeling involves interactions between several cell types, including epithelial cells, fibroblasts, endothelial cells, pericytes, smooth muscle cells, and both resident and recruited cells of the immune system. Where the remodeling process fails to repair, fibrosis occurs with the formation of scar tissue. In some instances, this remodeling is continuous and leads to inflammation and disease progression in the injured tissue.

Hedgehog (Hh) signaling is extensively involved in embryogenesis and many types of progenitor cells in various tissues express Patched (Ptc), the cell surface receptor for Hh ligands. In the absence of Hh ligand, the Ptc co-receptor Smoothened (Smo) is held in an inactive state by Ptc. When Hh ligand is present, it binds to Ptc, derepressing Smo, and permitting propagation of intracellular signals that culminate in the activation of target genes (e.g. Gli, Wnt, Bmp). Several components of the Hh signaling pathway, including Ptc and Gli, are themselves Hh-regulated genes, making Gli1 and Ptc gene expression one of the most reliable and robust measures of Hh pathway activation and inhibition.

While activation of the hedgehog pathway is often observed in cancer, the need exists for identifying new roles of this pathway in non-malignant diseases.

### SUMMARY

The invention discloses, at least in part, that a hedgehog (Hh) inhibitor, as a single agent or in combination, can reduce a fibrotic condition in a mammalian subject. In one embodiment, Applicants have shown that expression of a hedgehog ligand is localized in several fibrotic tissues, including heart, kidney and liver. In another embodiment, inhibition of hedgehog signaling using a Smoothened inhibitor, IPI-926, is shown to reduce pulmonary fibrosis, reduce body weight loss associated with fibrosis, and increase survival, of a pulmonary fibrosis animal model. In one embodiment, prophylactic treatment with a hedgehog inhibitor resulted in a significant decrease in fibrotic disease. The results described herein implicate hedgehog inhibition (e.g., treatment with IPI-926) as a useful target for ameliorating fibrotic conditions and disorders, including reducing fibrosis, and/or having a protective effect by decreasing weight loss associated with fibrosis and increasing survival. Experimental conditions for evaluating the anti-fibrotic effects of a hedgehog inhibitor in other animal fibrotic models, such as liver fibrosis, bone marrow fibrosis (e.g., myelofibrosis) and kidney fibrosis, are disclosed. Thus, methods and compositions for treating or preventing, fibrosis, or a fibrotic condition, by administering a hedgehog inhibitor, alone or in combination with other agents or therapeutic modalities, to a subject, e.g., a mammalian subject, are disclosed.

Accordingly, in one aspect, the present invention features a method of reducing fibrosis in a cell or tissue. The method includes contacting a fibrotic cell or tissue with a hedgehog inhibitor (e.g., as a single agent or in combination with another agent or therapeutic modality), in an amount sufficient to decrease or inhibit the fibrosis. In one embodiment, the method is carried out in vivo, for example, in a mammalian subject, e.g., an animal model or as part of therapeutic protocol. In one embodiment, the fibrosis includes a fibrotic condition.

In another aspect, the invention features a method of treating or preventing a fibrotic condition. The method includes administering a hedgehog inhibitor (e.g., as a single agent or in combination with another agent or therapeutic modality), to a subject in need thereof, in an amount sufficient to decrease or inhibit the fibrotic condition in the subject.

In certain embodiments, reducing fibrosis, or treatment of a fibrotic condition, includes reducing or inhibiting one or more of: formation or deposition of tissue fibrosis; reducing the size, cellularity (e.g., fibroblast or immune cell numbers); composition; cellular, collagen or hydroxyproline content, of a fibrotic lesion; expression or activity of a fibrogenic protein; or reducing fibrosis associated with an inflammatory response.

In certain embodiments, the fibrotic condition is primary fibrosis. In one embodiment, the fibrotic condition is idiopathic. In other embodiments, the fibrotic condition is associated with (e.g., is secondary to) a disease (e.g., an infectious disease, an inflammatory disease, an autoimmune disease, a malignant or cancerous disease, and/or a connective disease); a toxin; an insult (e.g., an environmental hazard (e.g., asbestos, coal dust, polycyclic aromatic hydrocarbons), cigarette smoking, a wound); a medical treatment (e.g., surgical incision, chemotherapy or radiation), or a combination thereof.

In certain embodiments, the fibrotic condition is a fibrotic condition of the lung, a fibrotic condition of the liver, a fibrotic condition of the heart or vasculature, a fibrotic condition of the kidney, a fibrotic condition of the skin, a fibrotic condition of the gastrointestinal tract, a fibrotic condition of the bone marrow or a hematopoietic tissue, a fibrotic condition of the nervous system, or a combination thereof.

In other embodiment, the fibrotic condition affects a tissue chosen from one or more of muscle, tendon, cartilage, skin (e.g., skin epidermis or endodermis), cardiac tissue, vascular tissue (e.g., artery, vein), pancreatic tissue, lung tissue, liver tissue, kidney tissue, uterine tissue, ovarian tissue, neural tissue, testicular tissue, peritoneal tissue, colon, small intestine, biliary tract, gut, bone marrow, or hematopoietic tissue.

In certain embodiments, the fibrotic condition is a fibrotic condition of the lung. In certain embodiments, the fibrotic condition of the lung is chosen from one or more of: pulmonary fibrosis, idiopathic pulmonary fibrosis (IPF), usual interstitial pneumonitis (UIP), interstitial lung disease, cryptogenic fibrosing alveolitis (CFA), or bronchiectasis. In one embodiment, the fibrosis of the lung is secondary to a disease, a toxin, an insult, a medical treatment, or a combination thereof. For example, the fibrosis of the lung can be associated with (e.g., secondary to) one or more of: a disease process such as asbestosis and silicosis; an occupational hazard; an environmental pollutant; cigarette smoking; an autoimmune connective tissue disorders (e.g., rheumatoid arthritis, scleroderma and systemic lupus erythematosus (SLE)); a connective tissue disorder such as sarcoidosis; an infectious disease, e.g., infection, particularly chronic infection; a medical treatment, including but not limited to, radiation therapy, and drug therapy, e.g., chemotherapy (e.g., treatment with as bleomycin, methotrexate, amiodarone, busulfan, and/or nitrofurantoin). In one embodiment, the fibrotic condition of the lung treated with the methods of the invention is associated with (e.g., secondary to) a cancer treatment, e.g., treatment of a cancer (e.g., squamous cell carcinoma, testicular cancer, Hodgkin's disease with bleomycin).

In certain embodiments, the fibrotic condition is a fibrotic condition of the liver. In certain embodiments, the fibrotic condition of the liver is chosen from one or more of: fatty liver disease, steatosis (e.g., nonalcoholic steatohepatitis (NASH), cholestatic liver disease (e.g., primary biliary cirrhosis (PBC)), cirrhosis, alcohol induced liver fibrosis, biliary duct injury, biliary fibrosis, or cholangiopathies. In other embodiments, hepatic or liver fibrosis includes, but is not limited to, hepatic fibrosis associated with alcoholism, viral infection, e.g., hepatitis (e.g., hepatitis C, B or D), autoimmune hepatitis, non-alcoholic fatty liver disease (NAFLD), progressive massive fibrosis, exposure to toxins or irritants (e.g., alcohol, pharmaceutical drugs and environmental toxins).

In certain embodiments, the fibrotic condition is a fibrotic condition of the heart. In certain embodiments, the fibrotic condition of the heart is myocardial fibrosis (e.g., myocardial fibrosis associated with radiation myocarditis, a surgical procedure complication (e.g., myocardial post-operative fibrosis), infectious diseases (e.g., Chagas disease, bacterial, trichinosis or fungal myocarditis)); granulomatous, metabolic storage disorders (e.g., cardiomyopathy, hemochromatosis); developmental disorders (e.g., endocardial fibroelastosis); arteriosclerotic, or exposure to toxins or irritants (e.g., drug induced cardiomyopathy, drug induced cardiotoxicity, alcoholic cardiomyopathy, cobalt poisoning or exposure). In certain embodiments, the myocardial fibrosis is associated with an inflammatory disorder of cardiac tissue (e.g., myocardial sarcoidosis).

In certain embodiments, the fibrotic condition is a fibrotic condition of the kidney. In certain embodiments, the fibrotic condition of the kidney is chosen from one or more of: renal fibrosis (e.g., chronic kidney fibrosis), nephropathies associated with injury/fibrosis (e.g., chronic nephropathies associated with diabetes (e.g., diabetic nephropathy)), lupus, scleroderma of the kidney, glomerular nephritis, focal segmental glomerular sclerosis, IgA nephropathyrenal fibrosis associated with human chronic kidney disease (CKD), chronic progressive nephropathy (CPN), tubulointerstitial fibrosis, ureteral obstruction, chronic uremia, chronic interstitial nephritis, radiation nephropathy, glomerulosclerosis, progressive glomerulonephrosis (PGN), endothelial/thrombotic microangiopathy injury, HIV-associated nephropathy, or fibrosis associated with exposure to a toxin, an irritant, or a chemotherapeutic agent.

In certain embodiments, the fibrotic condition is a fibrotic condition of the skin. In certain embodiments, the fibrotic condition of the skin is chosen from one or more of: skin fibrosis, scleroderma, nephrogenic systemic fibrosis (e.g., resulting after exposure to gadolinium (which is frequently used as a contrast substance for MRIs) in patients with severe kidney failure), and keloid.

In certain embodiments, the fibrotic condition is a fibrotic condition of the gastrointestinal tract. In certain embodiments, the fibrotic condition is chosen from one or more of fibrosis associated with scleroderma; radiation induced gut fibrosis; fibrosis associated with a foregut inflammatory disorder such as Barrett's esophagus and chronic gastritis, and/or fibrosis associated with a hindgut inflammatory disorder, such as inflammatory bowel disease (IBD), ulcerative colitis and Crohn's disease.

In certain embodiments, the fibrotic condition is a fibrotic condition of the bone marrow or a hematopoietic tissue. In certain embodiments, the fibrotic condition of the bone marrow is an intrinsic feature of a chronic myeloproliferative neoplasm of the bone marrow, such as primary myelofibrosis (also referred to herein as agnogenic myeloid metaplasia or chronic idiopathic myelofibrosis). In other embodiments, the bone marrow fibrosis is associated with (e.g., is secondary to) a malignant condition or a condition caused by a clonal proliferative disease. In other embodiments, the bone marrow fibrosis is associated with a hematologic disorder (e.g., a hematologic disorder chosen from one or more of polycythemia vera, essential thrombocythemia, myelodysplasia, hairy cell leukemia, lymphoma (e.g., Hodgkin or non-Hodgkin lymphoma), multiple myeloma or chronic myelogeneous leukemia (CML)). In yet other embodiments, the bone marrow fibrosis is associated with (e.g., secondary to) a non-hematologic disorder (e.g., a non-hematologic disorder chosen from solid tumor metastasis to bone marrow, an autoimmune disorder (e.g., systemic lupus erythematosus, scleroderma, mixed connective tissue disorder, or polymyositis), an infection (e.g., tuberculosis), or secondary hyperparathyroidism associated with vitamin D deficiency.

In certain embodiments, the fibrotic condition is found in a tissue exhibiting hedgehog activity. In one embodiment, the hedgehog inhibitor reduces or inhibits a hedgehog signaling pathway. For example, the hedgehog inhibitor reduces or inhibits the activity of a hedgehog receptor, e.g., Smoothened or Patched. In one embodiment, the hedgehog inhibitor is a Smoothened inhibitor or modulator.

In certain embodiments, the hedgehog inhibitor is the compound of formula (I):

or a pharmaceutically acceptable salt and/or solvate thereof; wherein R¹, R², R³, and R⁴ are as defined herein.

In certain embodiments, the compound of formula (I) is compound of formula 32 (also referred to herein as “IPI-926”):

or a pharmaceutically acceptable salt and/or solvate thereof. An example of a pharmaceutically acceptable salt of the compound of formula I is the hydrochloride salt.

In other embodiments, the hedgehog inhibitor is chosen from one or more of: GDC-0449 (also known as RG3616), BMS-833923, LDE-225, LEQ-506, PF-04449913, SMOi2-17, SANT 1,2, or MK-4101.

In other embodiments, one or more hedgehog inhibitors (e.g., one or more hedgehog inhibitors described herein) are administered in combination. In one embodiment, the hedgehog inhibitors are administered concurrently. In another embodiment the inhibitors are administered sequentially. For example, a combination of e.g., IPI-926 and a second hedgehog inhibitor, e.g., GDC-0449, can be administered concurrently or sequentially. In one embodiment, the second hedgehog inhibitor, e.g., GDC-0449, is administered first, followed, with or without a period of overlap, by administration of IPI-926. In another embodiment, IPI-926 is administered first, followed, with or without a period of overlap, by administration of the second hedgehog inhibitor, e.g., GDC-0449.

In one embodiment, the subject treated is a mammal, e.g., a primate, typically a human (e.g., a patient having, or at risk of having, a fibrotic condition as described herein). In another embodiment, the subject treated is in need of hedgehog inhibition (e.g., has been evaluated to show elevated hedgehog levels, a fibrotic marker, or a genetic abnormality as described herein). In other embodiments, the subject has a mutation associated with a disorder having a fibrotic component. For example, the subject is a patient having, or at risk of having, primary myelofibrosis having a gain-of-function mutation in a gene that regulates hematopoiesis, such as Janus kinase 2 (JAK2) (e.g., JAK2^{V617F}) or the thrombopoietin receptor (MPL). In other embodiments, the subject has, or is at risk of having, a mutation in Bcr-abl. In other embodiments, the subject has, or is at risk of having, a cytogenic abnormality associated with a malignancy or cancer (e.g., a cytogenic abnormality associated with a myeloplastic syndrome, hairy cell leukemia, a lymphoma or multiple myeloma). In other embodiments, the subject has, or is at risk of having, a SMAD mutation. Any of these mutations or abnormalities can be evaluating prior to, during, or after the course of therapy.

In some embodiments, the hedgehog inhibitor is administered as a pharmaceutical composition comprising the hedgehog inhibitor, or a pharmaceutically acceptable salt thereof, and a pharmaceutically acceptable excipient.

In certain embodiments, the hedgehog inhibitor is administered, or is present in the composition, e.g., the pharmaceutical composition.

The hedgehog inhibitors described herein can be administered to the subject systemically (e.g., orally, parenterally, subcutaneously, intravenously, rectally, intramuscularly, intraperitoneally, intranasally, transdermally, or by inhalation or intracavitary installation). Typically, the hedgehog inhibitors are administered orally.

In one embodiment, the hedgehog inhibitor is IPI-926. IPI-926 can be administered orally in a daily schedule at a dose of about 20 mg to 200 mg, alone or in combination with a second agent as described herein.

The methods and compositions of the invention can, optionally, be used in combination with other agents or therapeutic modalities, e.g., one or more agents, and/or in combination with surgical and/or radiation procedures. Any combination of the hedgehog inhibitor and other agents or therapeutic modalities can be used. The hedgehog inhibitor and other therapeutic modalities can be administered before treatment, concurrently with treatment, post-treatment, or during remission of the disorder. In one embodiment, the second agent is administered simultaneously or sequentially with the hedgehog inhibitor.

In other embodiments, the hedgehog inhibitor and the second agent are administered as separate compositions, e.g., pharmaceutical compositions. In other embodiments, the hedgehog inhibitor and the agent are administered separately, but via the same route (e.g., both orally or both intravenously). In still other instances, the hedgehog inhibitor and the agent are administered in the same composition, e.g., pharmaceutical composition.

In embodiments where a fibrotic condition of the heart is treated, the hedgehog inhibitor can be administered in combination with eplerenone, furosemide, pycnogenol, spironolactone, TcNC100692, torasemide (e.g., prolonged release form of torasemide), or a combination thereof.

In embodiments where a fibrotic condition of the kidney is treated, the hedgehog inhibitor can be administered in combination with cyclosporine, cyclosporine A, daclizumab, everolimus, gadofoveset trisodium (ABLAVAR®), imatinib mesylate (GLEEVEC®), matinib mesylate, methotrexate, mycophenolate mofetil, prednisone, sirolimus, spironolactone, STX-100, tamoxifen, TheraCLEC™, or a combination thereof.

In embodiments where a fibrotic condition of the skin is treated, the hedgehog inhibitor can be administered in combination with Bosentan (Tracleer), p144, pentoxifylline; pirfenidone; pravastatin, STI571, Vitamin E, or a combination thereof.

In embodiments where a fibrotic condition of the bone marrow is treated, the hedgehog inhibitor can be administered in combination with an agent chosen from a Jak2 inhibitor (including, but not limited to, INCB018424, XL019, TG101348, or TG101209), an immunomodulator, e.g., an IMID (including, but not limited to thalidomide, lenalidomide, or panolinomide), hydroxyurea, an androgen, erythropoietic stimulating agents, prednisone, danazol, HDAC inhibitors, or other agents or therapeutic modalities (e.g., stem cell transplants, or radiation).

In embodiments where a fibrotic condition of the gastrointestinal fibrosis is treated, the hedgehog inhibitor can be administered in combination with ALTU-135, bucelipase alfa (INN), DCI1020, EUR-1008 (ZENPEP™), ibuprofen, Lym-X-Sorb powder, pancrease MT, pancrelipase (e.g., pancrelipase delayed release), pentade canoic acid (PA), repaglinide, TheraCLEC™, triheptadecanoin (THA), ULTRASE MT20, ursodiol, or a combination thereof.

In other embodiments, the hedgehog inhibitor is administered in combination with one or more of allogeneic bone marrow transplant, erythropoietin, radiation, or a histone deacetylase inhibitor.

The methods of the invention can further include the step of monitoring the subject, e.g., for a change (e.g., an increase or decrease) in one or more of: fibrotic lesion size (e.g., Sirius red staining, collagen or hydroxyproline content); cellularity; composition; fibrogenic gene expression (e.g., Col1a1, Acta2, TIMP-1, Lox); alpha-SMA immunohistochemistry (IHC) and/or Western Blot; immune/inflammatory levels; hedgehog levels or signaling; stromal activation; levels of one or more markers (e.g., cancer markers); or any other parameter related to clinical outcome. In additional embodiments, the method further includes monitoring in a subject with bone marrow fibrosis one or more of: monitoring peripheral blood counts (e.g., red blood cells, white blood cells, platelets), wherein an increase in peripheral blood counts is indicative of an improved outcome. In other embodiments, the method further includes monitoring in a subject with bone marrow fibrosis one or more of: spleen size, liver size, and size of extramedullary hematopoiesis, wherein a decrease in one or more of these parameters is indicative of an improved outcome. The subject can be monitored in one or more of the following periods: prior to beginning of treatment; during the treatment; or after one or more elements of the treatment have been administered. Monitoring can be used to evaluate the need for further treatment with the same hedgehog inhibitor, alone or in combination with, another agent, or for additional treatment with additional agents. Generally, a decrease in one or more of the parameters described above is indicative of the improved condition of the subject.

The methods of the invention can further include the step of analyzing a nucleic acid or protein from the subject, e.g., analyzing the genotype of the subject. In one embodiment, a hedgehog protein, or a nucleic acid encoding a hedgehog ligand and/or an upstream or downstream component(s) of the hedgehog signaling, e.g., a receptor, activator or inhibitor of hedgehog, is analyzed. The elevated hedgehog ligand can be detected in blood, urine, circulating cells, a tissue biopsy or a bone marrow biopsy. The elevated hedgehog ligand can also be detected by systemic administration of a labeled form of an antibody to a hedgehog ligand followed by imaging. In other embodiments, the expression or activity of one or more hedgehog pathway responsive gene (e.g., Gli-1, Gli-2, PTCH, WIF-1, IGFBP3) can be evaluated.

In other embodiments, the method further includes analyzing a nucleic acid or a protein from a cancer marker, e.g., Janus kinase 2 (JAK2) (e.g., JAK2^{V617F}); the thrombopoietin receptor (MPL) for bone marrow fibrotic conditions, such as myeloproliferative neoplasms; a mutation in Bcr-abl; or a cytogenic abnormality associated with a malignancy.

The analysis can be used, e.g., to evaluate the suitability of, or to choose between alternative treatments, e.g., a particular dosage, mode of delivery, time of delivery, inclusion of adjunctive therapy, e.g., administration in combination with a second agent, or generally to determine the subject's probable drug response phenotype or genotype. The nucleic acid or protein can be analyzed at any stage of treatment, but preferably, prior to administration of the hedgehog inhibitor and/or agent, to thereby determine appropriate dosage(s) and treatment regimen(s) of the hedgehog inhibitor (e.g., amount per treatment or frequency of treatments) for prophylactic or therapeutic treatment of the subject.

In certain embodiments, the methods of the invention further include the step of detecting elevated hedgehog ligand in the subject, prior to, or after, administering a hedgehog inhibitor to the patient. The elevated hedgehog ligand can be detected in blood, urine, circulating cells, a tissue biopsy or a bone marrow biopsy. The elevated hedgehog ligand can also be detected by systemic administration of a labeled form of an antibody to a hedgehog ligand followed by imaging. The step of detecting elevated hedgehog ligand can include the steps of measuring hedgehog ligand in the patient prior to administration of the other therapy, measuring hedgehog ligand in the patient after administration of the other therapy, and determining if the amount of hedgehog ligand after administration of the other therapy is greater than the amount of hedgehog ligand before administration of the other therapy. The other therapy can be, for example, an anti-inflammatory, an anti-fibrotic, a chemotherapeutic, surgery or radiation therapy.

In another aspect, the invention features a composition, e.g., a pharmaceutical composition, that includes one or more hedgehog inhibitors, e.g., a hedgehog inhibitor as described herein, and one or more agents (e.g., an agent as disclosed herein). The composition can further include a pharmaceutically-acceptable carrier or excipient.

In another aspect, the invention features a composition for use, or the use, of a hedgehog inhibitor, alone or in combination with a second agent or therapeutic modality described herein for the treatment of a fibrotic condition, e.g., a fibrotic condition as described herein.

In another aspect, the invention features therapeutic kits that include the hedgehog inhibitor, alone or in combination with an agent described herein, and instructions for use the treatment of a fibrotic condition, e.g., a fibrotic condition as described herein.

All publications, patent applications, patents, and other references mentioned herein are incorporated by reference in their entirety.

Other features, objects, and advantages of the invention will be apparent from the description and drawings, and from the claims.

### DEFINITIONS

Definitions of specific functional groups and chemical terms are described in detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in, for example, Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Certain compounds of the present invention can comprise one or more asymmetric centers, and thus can exist in various isomeric forms, i.e., stereoisomers (enantiomers, diastereomers, cis-trans isomers, E/Z isomers, etc.). Thus, inventive compounds and pharmaceutical compositions thereof can be in the form of an individual enantiomer, diastereomer or other geometric isomer, or can be in the form of a mixture of stereoisomers. Enantiomers, diastereomers and other geometric isomers can be isolated from mixtures (including racemic mixtures) by any method known to those skilled in the art, including chiral high pressure liquid chromatography (HPLC) and the formation and crystallization of chiral salts or prepared by asymmetric syntheses; see, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972).

Carbon atoms, unless otherwise specified, can optionally be substituted with one or more substituents. The number of substituents is typically limited by the number of available valences on the carbon atom, and can be substituted by replacement of one or more of the hydrogen atoms that would be available on the unsubstituted group. Suitable substituents are known in the art and include, but are not limited to, alkyl, alkenyl, alkynyl, alkoxy, alkoxy, aryl, aryloxy, arylthio, aralkyl, heteroaryl, heteroaralkyl, cycloalkyl, heterocyclyl, halo, azido, hydroxyl, thio, alkthiooxy, amino, nitro, nitrile, imino, amido, carboxylic acid, aldehyde, carbonyl, ester, silyl, alkylthio, haloalkyl (e.g., perfluoroalkyl such as —CF₃), ═O, ═S, and the like.

When a range of values is listed, it is intended to encompass each value and sub-range within the range. For example, an alkyl group containing 1-6 carbon atoms (C₁₋₆ alkyl) is intended to encompass, C₁, C₂, C₃, C₄, C₅, C₆, C₁₋₆, C₂₋₆, C₃₋₆, C₄₋₆, C₅₋₆, C₁₋₅, C₂₋₅, C₃₋₅, C₄₋₅, C₁₋₄, C₂₋₄, C₃₋₄, C₁₋₃, C₂₋₃, and C₁₋₂ alkyl.

The term “alkyl,” as used herein, refers to saturated, straight- or branched-chain hydrocarbon radical containing between one and thirty carbon atoms. In certain embodiments, the alkyl group contains 1-20 carbon atoms. Alkyl groups, unless otherwise specified, can optionally be substituted with one or more substituents. In certain embodiments, the alkyl group contains 1-10 carbon atoms. In certain embodiments, the alkyl group contains 1-6 carbon atoms. In certain embodiments, the alkyl group contains 1-5 carbon atoms. In certain embodiments, the alkyl group contains 1-4 carbon atoms. In certain embodiments, the alkyl group contains 1-3 carbon atoms. In certain embodiments, the alkyl group contains 1-2 carbon atoms. In certain embodiments, the alkyl group contains 1 carbon atom. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like.

The term “alkenyl,” as used herein, denotes a straight- or branched-chain hydrocarbon radical having at least one carbon-carbon double bond by the removal of a single hydrogen atom, and containing between two and thirty carbon atoms. Alkenyl groups, unless otherwise specified, can optionally be substituted with one or more substituents. In certain embodiments, the alkenyl group contains 2-20 carbon atoms. In certain embodiments, the alkenyl group contains 2-10 carbon atoms. In certain embodiments, the alkenyl group contains 2-6 carbon atoms. In certain embodiments, the alkenyl group contains 2-5 carbon atoms. In certain embodiments, the alkenyl group contains 2-4 carbon atoms. In certain embodiment, the alkenyl group contains 2-3 carbon atoms. In certain embodiments, the alkenyl group contains 2 carbon atoms. Alkenyl groups include, for example, ethenyl, propenyl, butenyl, 1-methyl-2-buten-1-yl, and the like.

The term “alkynyl,” as used herein, denotes a straight- or branched-chain hydrocarbon radical having at least one carbon-carbon triple bond by the removal of a single hydrogen atom, and containing between two and thirty carbon atoms. Alkynyl groups, unless otherwise specified, can optionally be substituted with one or more substituents. In certain embodiments, the alkynyl group contains 2-20 carbon atoms. In certain embodiments, the alkynyl group contains 2-10 carbon atoms. In certain embodiments, the alkynyl group contains 2-6 carbon atoms. In certain embodiments, the alkynyl group contains 2-5 carbon atoms. In certain embodiments, the alkynyl group contains 2-4 carbon atoms. In certain embodiments, the alkynyl group contains 2-3 carbon atoms. In certain embodiments, the alkynyl group contains 2 carbon atoms. Representative alkynyl groups include, but are not limited to, ethynyl, 2-propynyl (propargyl), 1-propynyl, and the like.

The terms “cycloalkyl”, used alone or as part of a larger moiety, refer to a saturated monocyclic or bicyclic hydrocarbon ring system having from 3-15 carbon ring members. Cycloalkyl groups, unless otherwise specified, can optionally be substituted with one or more substituents. In certain embodiments, cycloalkyl groups contain 3-10 carbon ring members. In certain embodiments, cycloalkyl groups contain 3-9 carbon ring members. In certain embodiments, cycloalkyl groups contain 3-8 carbon ring members. In certain embodiments, cycloalkyl groups contain 3-7 carbon ring members. In certain embodiments, cycloalkyl groups contain 3-6 carbon ring members. In certain embodiments, cycloalkyl groups contain 3-5 carbon ring members. Cycloalkyl groups include, without limitation, cyclopropyl, cyclobutyl, cyclopentyl, cyclohexyl, cycloheptyl, and cyclooctyl. The term “cycloalkyl” also includes saturated hydrocarbon ring systems that are fused to one or more aryl or heteroaryl rings, such as decahydronaphthyl or tetrahydronaphthyl, where the point of attachment is on the saturated hydrocarbon ring.

The term “aryl” used alone or as part of a larger moiety (as in “aralkyl”), refers to an aromatic monocyclic and bicyclic hydrocarbon ring system having a total of 6-10 carbon ring members. Aryl groups, unless otherwise specified, can optionally be substituted with one or more substituents. In certain embodiments of the present invention, “aryl” refers to an aromatic ring system which includes, but not limited to, phenyl, biphenyl, naphthyl, anthrancyl and the like, which can bear one or more substituents. Also included within the scope of the term “aryl”, as it is used herein, is a group in which an aryl ring is fused to one or more non-aromatic rings, such as indanyl, phthalimidyl or tetrahydronaphthalyl, and the like, where the point of attachment is on the aryl ring.

The term “aralkyl” refers to an alkyl group, as defined herein, substituted by aryl group, as defined herein, wherein the point of attachment is on the alkyl group.

The term “heteroatom” refers to boron, phosphorus, selenium, nitrogen, oxygen, or sulfur, and includes any oxidized form of nitrogen or sulfur, and any quaternized form of abasic nitrogen.

The terms “heteroaryl” used alone or as part of a larger moiety, e.g., “heteroaralkyl”, refer to an aromatic monocyclic or bicyclic hydrocarbon ring system having 5-10 ring atoms wherein the ring atoms comprise, in addition to carbon atoms, from one to five heteroatoms. Heteroaryl groups, unless otherwise specified, can optionally be substituted with one or more substituents. When used in reference to a ring atom of a heteroaryl group, the term “nitrogen” includes a substituted nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaryl ring is fused to one or more aryl, cycloalkyl or heterocycloalkyl rings, wherein the point of attachment is on the heteroaryl ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl.

The term “heteroaralkyl” refers to an alkyl group, as defined herein, substituted by a heteroaryl group, as defined herein, wherein the point of attachment is on the alkyl group.

As used herein, the terms “heterocycloalkyl” or “heterocyclyl” refer to a stable non-aromatic 5-7 membered monocyclic hydrocarbon or stable non-aromatic 7-10 membered bicyclic hydrocarbon that is either saturated or partially unsaturated, and having, in addition to carbon atoms, one or more heteroatoms. Heterocycloalkyl or heterocyclyl groups, unless otherwise specified, can optionally be substituted with one or more substituents. When used in reference to a ring atom of a heterocycloalkyl group, the term “nitrogen” includes a substituted nitrogen. The point of attachment of a heterocycloalkyl group can be at any of its heteroatom or carbon ring atoms that results in a stable structure. Examples of heterocycloalkyl groups include, without limitation, tetrahydrofuranyl, tetrahydrothienyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. “Heterocycloalkyl” also include groups in which the heterocycloalkyl ring is fused to one or more aryl, heteroaryl or cycloalkyl rings, such as indolinyl, chromanyl, phenanthridinyl, or tetrahydroquinolinyl, where the radical or point of attachment is on the heterocycloalkyl ring.

The term “unsaturated”, as used herein, means that a moiety has one or more double or triple bonds.

As used herein, the term “partially unsaturated” refers to a ring moiety that includes at least one double or triple bond. The term “partially unsaturated” is intended to encompass rings having multiple sites of unsaturation, but is not intended to include aromatic groups, such as aryl or heteroaryl moieties, as defined herein.

The term “diradical” as used herein refers to an alkyl, alkenyl, alkynyl, aryl, cycloalkyl, heterocycloalkyl, aralkyl, heteroaryl, and heteroaralkyl groups, as described herein, wherein 2 hydrogen atoms are removed to form a divalent moiety. Diradicals are typically end with a suffix of “-ene”. For example, alkyl diradicals are referred to as alkylenes (for example:

and —(CR′₂)ₓ— wherein R′ is hydrogen or other substituent and x is 1, 2, 3, 4, 5 or 6); alkenyl diradicals are referred to as “alkenylenes”; alkynyl diradicals are referred to as “alkynylenes”; aryl and aralkyl diradicals are referred to as “arylenes” and “aralkylenes”, respectively (for example:

heteroaryl and heteroaralkyl diradicals are referred to as “heteroarylenes” and “heteroaralkylenes”, respectively (for example:

cycloalkyl diradicals are referred to as “cycloalkylenes”; heterocycloalkyl diradicals are referred to as “heterocycloalkylenes”; and the like.

The terms “halo”, “halogen” and “halide” as used herein refer to an atom selected from fluorine (fluoro, F), chlorine (chloro, Cl), bromine (bromo, Br), and iodine (iodo, I).

As used herein, the term “haloalkyl” refers to an alkyl group, as described herein, wherein one or more of the hydrogen atoms of the alkyl group is replaced with one or more halogen atoms. In certain embodiments, the haloalkyl group is a perhaloalkyl group, that is, having all of the hydrogen atoms of the alkyl group replaced with halogens (e.g., such as the perfluoroalkyl group —CF₃).

As used herein, the term “azido” refers to the group —N₃.

As used herein, the term “nitrile” refers to the group —CN.

As used herein, the term “nitro” refers to the group —NO₂.

As used herein, the term “hydroxyl” or “hydroxy” refers to the group —OH.

As used herein, the term “thiol” or “thio” refers to the group —SH.

As used herein, the term “carboxylic acid” refers to the group —CO₂H.

As used herein, the term “aldehyde” refers to the group —CHO.

As used herein, the term “alkoxy” refers to the group —OR′, wherein R′ is an alkyl, alkenyl or alkynyl group, as defined herein.

As used herein, the term “aryloxy” refers to the group —OR′, wherein each R′ is an aryl or heteroaryl group, as defined herein.

As used herein, the term “alkthiooxy” refers to the group —SR′, wherein each R′ is, independently, a carbon moiety, such as, for example, an alkyl, alkenyl, or alkynyl group, as defined herein.

As used herein, the term “arylthio” refers to the group —SR′, wherein each R′ is an aryl or heteroaryl group, as defined herein.

As used herein, the term “amino” refers to the group —NR′₂, wherein each R′ is, independently, hydrogen, a carbon moiety, such as, for example, an alkyl, alkenyl, alkynyl, aryl or heteroaryl group, as defined herein, or two R′ groups together with the nitrogen atom to which they are bound form a 5-8 membered ring.

As used herein, the term “carbonyl” refers to the group —C(═O)R′, wherein R′ is, independently, a carbon moiety, such as, for example, an alkyl, alkenyl, alkynyl, aryl or heteroaryl group, as defined herein.

As used herein, the term “ester” refers to the group —C(═O)OR′ or —OC(═O)R′ wherein each R′ is, independently, a carbon moiety, such as, for example, an alkyl, alkenyl, alkynyl, aryl or heteroaryl group, as defined herein.

As used herein, the term “amide” or “amido” refers to the group —C(═O)N(R′)₂ or —NR′C(═O)R′ wherein each R′ is, independently, hydrogen or a carbon moiety, such as, for example, an alkyl, alkenyl, alkynyl, aryl or heteroaryl group, as defined herein, or two R′ groups together with the nitrogen atom to which they are bound form a 5-8 membered ring.

The term “sulfonamido” or “sulfonamide” refers to the group —N(R′)SO₂R′ or —SO₂N(R′)₂, wherein each R′ is, independently, hydrogen or a carbon moiety, such as, for example, an alkyl, alkenyl, alkynyl, aryl or heteroaryl group, as defined herein, or two R′ groups together with the nitrogen atom to which they are bound form a 5-8 membered ring.

The term “sulfamido” or “sulfamide” refers to the group —NR′SO₂N(R′)₂, wherein each R′ is, independently, hydrogen or a carbon moiety, such as, for example, an alkyl, alkenyl, alkynyl, aryl or heteroaryl group, as defined herein, or two R′ groups together with the nitrogen atom to which they are bound form a 5-8 membered ring.

As used herein, the term “imide” or “imido” refers to the group —C(═NR′)N(R′)₂ or —NR′C(═NR′)R′ wherein each R′ is, independently, hydrogen or a carbon moiety, such as, for example, an alkyl, alkenyl, alkynyl, aryl or heteroaryl group, as defined herein, or wherein two R′ groups together with the nitrogen atom to which they are bound form a 5-8 membered ring.

As used herein “silyl” refers to the group —SiR′ wherein R′ is a carbon moiety, such as, for example, an alkyl, alkenyl, alkynyl, aryl or heteroaryl group.

In some cases, the hedgehog inhibitor can contain one or more basic functional groups (e.g., such as an amino group), and thus is capable of forming pharmaceutically acceptable salts with pharmaceutically acceptable acids. The term “pharmaceutically acceptable salts” in these instances refers to the relatively non-toxic, inorganic and organic acid addition salts. These salts can be prepared in situ in the administration vehicle or the dosage form manufacturing process, or by separately treating the compound in its free base form with a suitable acid. Examples of pharmaceutically acceptable, nontoxic acid addition salts from inorganic acids include, but are not limited to, hydrochloric, hydrobromic, phosphoric, sulfuric, nitric and perchloric acid or from organic acids include, but are not limited to, acetic, adipic, alginic, ascorbic, aspartic, 2-acetoxybenzoic, benzenesulfonic, benzoic, bisulfonic, boric, butyric, camphoric, camphorsulfonic, citric, cyclopentanepropionic, digluconic, dodecylsulfonic, ethanesulfonic, 1,2-ethanedisulfonic, formic, fumaric, glucoheptonic, glycerophosphonic, gluconic, hemisulfonic, heptanoic, hexanoic, hydroiodic, 2-hydroxyethanesulfonic, hydroxymaleic, isothionic, lactobionic, lactic, lauric, lauryl sulfonic, malic, maleic, malonic, methanesulfonic, 2-naphthalenesulfonic, napthylic, nicotinic, oleic, oxalic, palmitic, pamoic, pectinic, persulfonic, 3-phenylpropionic, picric, pivalic, propionic, phenylacetic, stearic, succinic, salicyclic, sulfanilic, tartaric, thiocyanic, p-toluenesulfonic, undecanoic, and valeric acid addition salts, and the like. In other cases, the hedgehog inhibitor can contain one or more acidic functional groups, and thus is capable of forming pharmaceutically acceptable salts with pharmaceutically acceptable bases. The term “pharmaceutically acceptable salts” in these instances refers to the relatively non-toxic, inorganic and organic base addition salts. These salts can likewise be prepared in situ in the administration vehicle or the dosage form manufacturing process, or by separately treating the compound in its free acid form with a suitable base. Examples of suitable bases include, but are not limited to, metal hydroxides, metal carbonates or metal bicarbonates, wherein the metal is an alkali or alkaline earth metal such as lithium, sodium, potassium, calcium, magnesium, or aluminum. Suitable bases can also include ammonia or organic primary, secondary or tertiary amines. Representative organic amines useful for the formation of base addition salts include, for example, ethylamine, diethylamine, ethylenediamine, ethanolamine, diethanolamine, piperazine and the like (see, e.g., Berge et al., supra).

The term “solvate” refers to a compound of the present invention having either a stoichiometric or non-stoichiometric amount of a solvent associated with the compound. The solvent can be water (i.e., a hydrate), and each molecule of inhibitor can be associated with one or more molecules of water (e.g., monohydrate, dihydrate, trihydrate, etc.). The solvent can also be an alcohol (e.g., methanol, ethanol, propanol, isopropanol, etc.), a glycol (e.g., propylene glycol), an ether (e.g., diethyl ether), an ester (e.g., ethyl acetate), or any other suitable solvent. The hedgehog inhibitor can also exist as a mixed solvate (i.e., associated with two or more different solvents).

The term “sugar” as used herein refers to a natural or an unnatural monosaccharide, disaccharide or oligosaccharide comprising one or more pyranose or furanose rings. The sugar can be covalently bonded to the steroidal alkaloid of the present invention through an ether linkage or through an alkyl linkage. In certain embodiments the saccharide moiety can be covalently bonded to a steroidal alkaloid of the present invention at an anomeric center of a saccharide ring. Sugars can include, but are not limited to ribose, arabinose, xylose, lyxose, allose, altrose, glucose, mannose, gulose, idose, galactose, talose, glucose, and trehalose.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

### DESCRIPTION OF THE FIGURES

The application file contains at least one drawing executed in color. Copies of this patent application publication with color drawing(s) will be provided by the Office upon request and payment of the necessary fee.

FIG. 1 depicts the mean percent weight change in a bleomycin-induced pulmonary fibrosis model in C57BL/6 mice±treatment with IPI926. Animals were weighed daily, the percent weight change from day 0 was calculated, and group means and standard errors of the mean (SEM) calculated for each day.

FIG. 2 is a graph showing weight change and the area under the curve for untreated mice and IPI-926-treated mice in a mouse model of pulmonary fibrosis. The area under the curve (AUC) was calculated for the percent weight change exhibited by each animal in the study. This calculation was made using the trapezoidal rule transformation. Group means are shown with error bars representing SEM for each group. A One Way ANOVA was performed to compare differences between groups. Significant differences were observed between the vehicle treated control group and the groups treated with IPI-926 on Days −10 to 30 or Days 0 to 30 (P<0.001 for both comparisons).

FIG. 3 is a bar graph depicting Total White Blood Cell (WBC) Counts in bronchoalveloar lavage fluid (BAL) in a mouse model of pulmonary fibrosis±treatment with IPI926. Total white counts were measured using an automated hematology analyzer and group means and standard errors of the mean (SEM) calculated for each day.

FIG. 4 is a bar graph depicting Differential Lymphocyte Counts in BAL obtained from a mouse model of pulmonary fibrosis in untreated mice or in mice treated with IPI-926. Percentage lymphocyte counts were measured using an automated hematology analyzer and group means and standard errors of the mean (SEM) calculated for each day.

FIG. 5 is a bar graph depicting Differential Neutrophil Counts in BAL obtained from a mouse model of pulmonary fibrosis in untreated mice or in mice treated with IPI-926. Percentage neutrophil counts were measured using an automated hematology analyzer and group means and standard errors of the mean (SEM) calculated for each day.

FIG. 6 is a bar graph showing Differential Monocyte Counts in BAL obtained from untreated mice or mice treated with IPI-926 in a mouse model of pulmonary fibrosis. Percentage monocyte counts were measured using an automated hematology analyzer and group means and standard errors of the mean (SEM) calculated for each day.

FIG. 7 is a bar graph depicting the Chronic Inflammation Scores for lung sections in Example 2. Chronic inflammation is increased in the Vehicle group compared to the Naive group (p<0.05), indicating Bleomycin-induced pulmonary damage. All three IPI-926 treatments reduce inflammation compared to the Vehicle control group. The IPI-926 dosing regimen from Day 0 to Day 30 shows the greatest effect compared to the Vehicle control group. Lung tissue was analyzed at day 30. Statistical analysis was performed according to the Mann-Whitney test.

FIG. 8 is a bar graph showing Interstitial Fibrosis Scores for lung sections in Example 2. Interstitial fibrosis is increased in the Vehicle group compared to the Naive group (p<0.01), indicating Bleomycin-induced pulmonary damage. All three IPI-926 treatments reduce inflammation compared to the Vehicle control group. The IPI-926 dosing regimen from Day-10 to Day 30 shows the greatest effect compared to the Vehicle control group. Statistical analysis was performed according to the Mann-Whitney test.

FIG. 9 is a bar graph summarizing the number of foci of interstitial fibrosis that fill a 10× objective field for lung sections in Example 2. The number of foci of interstitial fibrosis is increased in the Vehicle group compared to the Naive group (p<0.01), indicating bleomycin-induced pulmonary damage. All three IPI-926 treatments reduce inflammation compared to the Vehicle control group. The IPI-926 dosing regimen from Day-10 to Day 30 shows the greatest effect compared to the Vehicle control group. Statistical analysis was performed according to the Mann-Whitney test.

FIGS. 10A-10E show representative photomicrographs of lung samples from study INF-01, described herein in Example 2. Panels A-E correspond to Groups 1-5, respectively. Arrows point to areas of interstitial fibrosis. Trichrome stain; 40× magnification.

FIGS. 11A-11E are representative photomicrographs of lung samples from Example 2 as stained using PicroSiruis Red to stain connective tissues. Panels A-E correspond to Naïve, Vehicle, and treated samples for 10, 0 and 14 days, respectively.

FIGS. 12A-12E are representative photomicrographs of lung samples from Example 2 as stained using a murine sonic hedgehog (SHH) antibody. Panels A-E correspond to Naïve, Vehicle, and treated samples for 10, 0 and 14 days, respectively.

FIGS. 13A-13E are representative photomicrographs of lung samples from Example 2 as stained using a murine GLi1 antibody. Panels A-E correspond to Naïve, Vehicle, and treated samples for 10, 0 and 14 days, respectively.

FIG. 14 is a bar graph showing the effect of IPI-926 treatment on bleomycin-induced pulmonary fibrotic mice. IPI-926 treatment was begun on the day of bleomycin insult, as assessed by ug of hydroxproline per g of animal body weight. SAL=saline; BLM=bleomycin insult.

FIG. 15 is a linear graph showing the effect of IPI-926 on body weight in murine bleomycin-induced pulmonary fibrosis.

FIG. 16 is a graph depicting the effect in animal survival of IPI-926 treatment on bleomycin-induced pulmonary fibrotic mice.

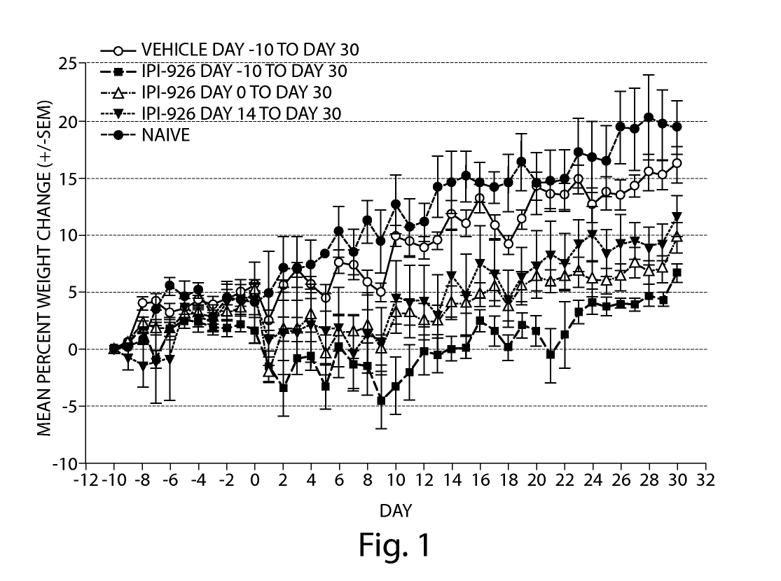

### SEQUENCE LISTING

Human Sonic Hedgehog protein (hSHH): APHNDSATGEPEASSG (SEQ ID NO. 1).

### DETAILED DESCRIPTION

The present invention provides a method of treating, fibrosis, or a fibrotic condition comprising administering a therapeutically effective amount of a hedgehog inhibitor, alone or in combination with a second agent, to a subject in need thereof. In one embodiment, the hedgehog inhibitor inhibits or reduces Hedgehog pathway signaling by inhibiting a hedgehog receptor. In one embodiment, the hedgehog inhibitor is an inhibitor of Smoothened. In one embodiment, the hedgehog inhibitor is IPI-926 (also referred to herein as “Compound 32”).

In one embodiment, the present application discloses localized hedgehog ligand expression in several fibrotic tissues, including heart, kidney and liver (Example 1). Given the immunohistochemical staining results described herein, Applicants believe that SHH ligand produced by parenchymal cells of an organ, such as kidney, liver or cardiac muscle, stimulate fibrosis in the stromal cells of that same organ.

In another embodiment, Applicants have shown that inhibition of hedgehog signaling using a Smoothened inhibitor, IPI-926, reduces pulmonary fibrosis, body weight loss associated with fibrosis, and increases survival, of a model of bleomycin-induced pulmonary fibrosis (see Example 2 below). In one embodiment, prophylactic treatment with a hedgehog inhibitor resulted in significant decreases in fibrotic lung disease.

In other embodiments, inhibition of hedgehog signaling is believed to reduce liver fibrosis in animal models (Example 3).

In other embodiments, Applicants have demonstrated that hedgehog (Hh) signaling between pancreatic tumor cells and the surrounding stroma plays a role in fibrosis associated with pancreatic cancer. For example, administration of hedgehog inhibitors, e.g., IPI-926, has a dramatic effect on the desmoplastic component in pancreatic animal models. Hh ligand dependent activation of the Hh pathway is believed to occur in myelofibrosis, either through ligand expressed by the abnormal stem cells or in the surrounding stroma or both Inhibition of Hedgehog pathway signaling by an inhibitor of Smoothened can result in decreased fibrosis, as seen in nonclinical models of pancreatic cancer (see Example 4).

These results described herein implicate hedgehog inhibition as a useful target for treating fibrotic conditions and disorders. Exemplary fibrotic conditions and disorders that can be treated with hedgehog inhibitors include, but are not limited to, liver fibrosis, such as liver fibrosis associated with liver injury (e.g., liver injury caused by alcohol, viral infection (e.g., Hepatitis B and C infection); pulmonary fibrosis (e.g., lung fibrosis caused by smoking, drugs such as bleomycin); cardiac fibrosis; bone marrow fibrosis, and kidney fibrosis.

As used herein, “fibrotic condition” refers to a disease or condition involving the formation and/or deposition of fibrous tissue, e.g., excessive connective tissue builds up in a tissue and/or spreads over or replaces normal organ tissue (reviewed in, e.g., Wynn, Nature Reviews 4:583-594 (2004) and Abdel-Wahab, O. et al. (2009) Annu. Rev. Med. 60:233-45, incorporated herein by reference). In certain embodiments, the fibrotic condition involves excessive collagen mRNA production and deposition. In certain embodiments, the fibrotic condition is caused, at least in part, by injury, e.g., chronic injury (e.g., an insult, a wound, a toxin, a disease). In certain embodiments, the fibrotic condition is associated with aberrant hedgehog signaling. In certain embodiments, the fibrotic condition is associated with an inflammatory, an autoimmune or a connective tissue disorder. For example, chronic inflammation in a tissue can lead to fibrosis in that tissue. Exemplary fibrotic tissues include, but are not limited to, biliary tissue, liver tissue, lung tissue, heart tissue, vascular tissue, kidney tissue, skin tissue, gut tissue, peritoneal tissue, bone marrow, and the like. In certain embodiments, the tissue is epithelial tissue.

As used herein, the term “patient” or “subject” refers to an animal, typically a human (i.e., a male or female of any age group, e.g., a pediatric patient (e.g., infant, child, adolescent) or adult patient (e.g., young adult, middle-aged adult or senior adult) or other mammal, such as a primate (e.g., cynomolgus monkey, rhesus monkey); other mammals such as rodents (mice, rats), cattle, pigs, horses, sheep, goats, cats, dogs; and/or birds, that will be or has been the object of treatment, observation, and/or experiment. When the term is used in conjunction with administration of a compound or drug, then the patient has been the object of treatment, observation, and/or administration of the compound or drug.

“Treating,” “treat,” and “treatment” as used herein, refers to partially or completely inhibiting or reducing the fibrotic condition which the subject is suffering. In one embodiment, this term refers to an action that occurs while a patient is suffering from, or is diagnosed with, the fibrotic condition, which reduces the severity of the condition, or retards or slows the progression of the condition. Treatment need not result in a complete cure of the condition; partial inhibition or reduction of the fibrotic condition is encompassed by this term.

“Therapeutically effective amount,” as used herein, refers to a minimal amount or concentration of a hedgehog inhibitor that, when administered alone or in combination, is sufficient to provide a therapeutic benefit in the treatment of the condition, or to delay or minimize one or more symptoms associated with the condition. The term “therapeutically effective amount” can encompass an amount that improves overall therapy, reduces or avoids symptoms or causes of the condition, or enhances the therapeutic efficacy of another therapeutic agent. The therapeutic amount need not result in a complete cure of the condition; partial inhibition or reduction of the fibrotic condition is encompassed by this term.

As used herein, unless otherwise specified, the terms “prevent,” “preventing” and “prevention” refers to an action that occurs before the subject begins to suffer from the condition, or relapse of such condition. The prevention need not result in a complete prevention of the condition; partial prevention or reduction of the fibrotic condition is encompassed by this term.

As used herein, unless otherwise specified, a “prophylactically effective amount” of a hedgehog inhibitor that, when administered alone or in combination, prevent the condition, or one or more symptoms associated with the condition, or prevent its recurrence. The term “prophylactically effective amount” can encompass an amount that improves overall prophylaxis or enhances the prophylactic efficacy of another prophylactic agent. The prophylactic amount need not result in a complete prevention of the condition; partial prevention or reduction of the fibrotic condition is encompassed by this term.

Exemplary fibrotic conditions that can be treated or prevented using the methods of the invention include, but are not limited to, a fibrotic condition of the lung, liver, heart, vasculature, kidney, skin, gastrointestinal tract, bone marrow, or a combination thereof. Each of these conditions is described in more detail herein.

Fibrosis of the lung (also referred to herein as “pulmonary fibrosis”) is characterized by the formation of scar tissue within the lungs, which results in a decreased function. Pulmonary fibrosis is associated with shortness of breath, which progresses to discomfort in the chest weakness and fatigue, and ultimately to loss of appetite and rapid weight-loss. Approximately 500,000 people in the U.S. and 5 million worldwide suffer from pulmonary fibrosis, and 40,000 people in the U.S. die annually from the disease. Pulmonary fibrosis has a number of causes, including radiation therapy, but can also be due to smoking or hereditary factors (Meltzer, E B et al. (2008) Orphanet J. Rare Dis. 3:8).

Applicants have shown in Example 2 below that inhibition of hedgehog signaling using a Smoothened inhibitor, IPI-926, reduced pulmonary fibrosis and associated inflammation. Other investigators have found that the Hh signal is upregulated, in part, from epithelial cells of the injured tissue, fibroblastic cells, and from infiltrating lymphocytes in chronic lung fibrosis (Stewart et al., “Expression of the Developmental Sonic Hedgehog (Shh) Signaling Pathway is Up-Regulated in Chronic Lung Fibrosis and the Shh Receptor Patched I is Present in Circulating T Lymphocytes” J. Pathology (2003) 199:488-495); and in pulmonary fibrosis (e.g., idiopathic pulmonary fibrosis (IPF), usual interstitial pneumonitis (UIP)), bronchiectasis, and interstitial lung disease (e.g., cryptogenic fibrosing alveolitis (CFA)) (see Coon et al., “Differential Epithelial Expression of SHH and FOXF1 in Usual and Nonspecific Interstitial Pneumonia” Exp. Mol. Pathol. (2006) 80:118-123; Selman et al., “Idiopathic Pulmonary Fibrosis: Aberrant Recapitulation of Developmental Programs?” PLoS Medicine (2008) 5:373-380).

Pulmonary fibrosis can occur as a secondary effect in disease processes such as asbestosis and silicosis, and is known to be more prevalent in certain occupations such as coal miner, ship workers and sand blasters where exposure to environmental pollutants is an occupational hazard (Green, F H et al. (2007) Toxicol Pathol. 35:136-47). Other factors that contribute to pulmonary fibrosis include cigarette smoking, and autoimmune connective tissue disorders, like rheumatoid arthritis, scleroderma and systemic lupus erythematosus (SLE) (Leslie, K O et al. (2007) Semin Respir Crit. Care Med. 28:369-78; Swigris, J J et al. (2008) Chest. 133:271-80; and Antoniou, K M et al. (2008) Curr Opin Rheumatol. 20:686-91). Other connective tissue disorders such as sarcoidosis can include pulmonary fibrosis as part of the disease (Paramothayan, S et al. (2008) Respir Med. 102:1-9), and infectious diseases of the lung can cause fibrosis as a long term consequence of infection, particularly chronic infections. Pulmonary fibrosis can also be a side effect of certain medical treatments, particularly radiation therapy to the chest and certain medicines like bleomycin, methotrexate, amiodarone, busulfan, and nitrofurantoin (Catane, R et al. (1979) Int J Radiat Oncol Biol Phys. 5:1513-8; Zisman, D A et al. (2001) Sarcoidosis Vasc Diffuse Lung Dis. 18:243-52; Rakita, L et al. (1983) Am Heart J. 106:906-16; Twohig, K J et al. (1990) Clin Chest Med. 11:31-54; and Witten C M. (1989) Arch Phys Med. Rehabil. 70:55-7). In other embodiments, idiopathic pulmonary fibrosis can occur where no clear causal agent or disease can be identified. Increasingly, it appears that genetic factors can play a significant role in these cases of pulmonary fibrosis (Steele, M P et al. (2007) Respiration 74:601-8; Brass, D M et al. (2007) Proc Am Thorac Soc. 4:92-100 and du Bois R M. (2006) Semin Respir Crit. Care Med. 27:581-8).

In certain embodiments, the fibrotic condition of the lung is chosen from one or more of: pulmonary fibrosis, idiopathic pulmonary fibrosis (IPF), usual interstitial pneumonitis (UIP), interstitial lung disease, cryptogenic fibrosing alveolitis (CFA), or bronchiectasis.

In other embodiments, pulmonary fibrosis includes, but is not limited to, pulmonary fibrosis associated with chronic obstructive pulmonary disease (COPD), scleroderma, pleural fibrosis, chronic asthma, acute lung syndrome, amyloidosis, bronchopulmonary dysplasia, Caplan's disease, Dressler's syndrome, histiocytosis X, idiopathic pulmonary haemosiderosis, lymphangiomyomatosis, mitral valve stenosis, polymyositis, pulmonary edema, pulmonary hypertension (e.g., idiopathic pulmonary hypertension (IPH)), pneumoconiosis, radiotherapy (e.g., radiation induced fibrosis), rheumatoid disease, Shaver's disease, systemic lupus erythematosus, systemic sclerosis, tropical pulmonary eosinophilia, tuberous sclerosis, Weber-Christian disease, Wegener's granulomatosis, Whipple's disease, or exposure to toxins or irritants (e.g., pharmaceutical drugs such as amiodarone, bleomycin, busulphan, carmustine, chloramphenicol, hexamethonium, methotrexate, methysergide, mitomycin C, nitrofurantoin, penicillamine, peplomycin, and practolol; inhalation of talc or dust, e.g., coal dust, silica). In certain embodiments, the pulmonary fibrosis is associated with an inflammatory disorder of the lung, e.g., asthma, COPD.

Hedgehog signaling has also been implicated in fibrotic conditions of the liver (also referred to herein as “hepatic fibrosis”), such as fatty liver disease e.g., steatosis such as nonalcoholic steatohepatitis (NASH) (see Fleig et al., “Hepatic Accumulation of Hedgehog-Reactive Progenitors Increases with Severity of Fatty Liver Damage in Mice” Lab. Invest. (2007) 87:1227-1239), biliary fibrosis, cholestatic liver disease (e.g., primary biliary cirrhosis (PBC), and cholangiopathies (e.g., chronic cholangiopathies)) (see Jung et al., “Bile Ductules and Stromal Cells Express Hedgehog Ligands and/or Hedgehog Target Genes in Primary Biliary Cirrhosis” Hepatology (2007) 45:1091-1096; Greenbaum, “Hedgehog Signaling in Biliary Fibrosis” J. Clin. Invest. (2008) 118:3263-3265; Omenetti et al., “Hedgehog-Mediated Mesenchymal-Epithelial Interactions Modulate Hepatic Response to Bile Duct Ligation” Lab. Invest. (2007) 87:499-514 (Omenetti 2007); Omenetti et al., “Hedgehog Signaling Regulates Epithelial-Mesenchymal Transition During Biliary Fibrosis in Rodents and Humans” J. Clin. Invest. (2008) 118: 3331-3342 (Omenetti 2008a); Omenetti et al., “The Hedgehog Pathway Regulates Remodeling Responses in Biliary Obstruction in Rats” Gut (2008) 57:1275-1282 (Omenetti 2008b); Omenetti and Diel, “The Adventures of Sonic Hedgehog in Development and Repair. II. Sonic Hedgehog and Liver Development, Inflammation, and Cancer” Am. J. Physiol. Gastrointest. Liver Physiol. (2008) 294:G595-G598 (Omenetti 2008c); Omenetti et al., “Repair-Related Activation of Hedgehog Signaling Promotes Cholangiocyte Chemokine Production” Hepatology (2009) 50:518-527 (Omenetti 2009)).

In certain embodiments, the fibrotic of the liver or hepatic fibrosis is chosen from one or more of: fatty liver disease, steatosis (e.g., nonalcoholic steatohepatitis (NASH), cholestatic liver disease, primary biliary cirrhosis (PBC), biliary fibrosis, cirrhosis, alcohol induced liver fibrosis, biliary duct injury, infection or viral induced liver fibrosis, congenital hepatic fibrosis, autoimmune hepatitis, or cholangiopathies (e.g., chronic cholangiopathies).

In certain embodiments, hepatic or liver fibrosis includes, but is not limited to, hepatic fibrosis associated with alcoholism, viral infection, e.g., hepatitis (e.g., hepatitis C, B or D), autoimmune hepatitis, non-alcoholic fatty liver disease (NAFLD), progressive massive fibrosis, exposure to toxins or irritants (e.g., alcohol, pharmaceutical drugs and environmental toxins such as arsenic), alpha-1 antitrypsin deficiency, hemochromatosis, Wilson's disease, galactosemia, or glycogen storage disease. In certain embodiments, the hepatic fibrosis is associated with an inflammatory disorder of the liver.

Hedgehog signaling has also been implicated in fibrotic conditions of the heart or vasculature, such as myocardial fibrosis. It has been suggested that while Hh signaling can assist in normal tissue-mediated recovery process, excessive amounts of endogenous Hh ligand exacerbates recovery and leads to fibrosis (see Bijlsma et al., (2008) “Endogenous Hedgehog Expression Contributes to Myocardial Ischemia-Reperfusion-Induced Injury” Exp. Biol. Med. 233:989-996). A fibrotic condition of the heart or vasculature includes, but is not limited to, myocardial fibrosis (e.g., myocardial fibrosis associated with radiation myocarditis, a surgical procedure complication (e.g., myocardial post-operative fibrosis), vascular restenosis, atherosclerosis, cerebral disease, peripheral vascular disease, infectious diseases (e.g., Chagas disease, bacterial, trichinosis or fungal myocarditis)); granulomatous, metabolic storage disorders (e.g., cardiomyopathy, hemochromatosis); developmental disorders (e.g, endocardial fibroelastosis); arteriosclerotic, or exposure to toxins or irritants (e.g., drug induced cardiomyopathy, drug induced cardiotoxicity, alcoholic cardiomyopathy, cobalt poisoning or exposure). In certain embodiments, the myocardial fibrosis is associated with an inflammatory disorder of cardiac tissue (e.g., myocardial sarcoidosis).

Hedgehog signaling has also been implicated in fibrotic conditions of the kidney, such as renal fibrosis (e.g., chronic kidney fibrosis) (see Fleig supra). The condition of renal fibrosis includes, but is not limited to, nephropathies associated with injury/fibrosis (e.g., chronic nephropathies associated with diabetes (e.g., diabetic nephropathy)), lupus, scleroderma of the kidney, glomerular nephritis, focal segmental glomerular sclerosis, IgA nephropathyrenal fibrosis associated with human chronic kidney disease (CKD), chronic kidney fibrosis, nephrogenic systemic fibrosis, chronic progressive nephropathy (CPN), tubulointerstitial fibrosis, ureteral obstruction (e.g., fetal partial urethral obstruction), chronic uremia, chronic interstitial nephritis, radiation nephropathy, glomerulosclerosis (e.g., focal segmental glomerulosclerosis (FSGS)), progressive glomerulonephrosis (PGN), endothelial/thrombotic microangiopathy injury, scleroderma of the kidney, HIV-associated nephropathy (HIVVAN), or exposure to toxins, irritants, chemotherapeutic agents. In one embodiment, the kidney fibrosis is mediated by a bone morphogeneic protein (BMP). In certain embodiments, the renal fibrosis is a result of an inflammatory disorder of the kidney.

In certain embodiments, the fibrotic condition is a fibrotic condition of the bone marrow. In certain embodiments, the fibrotic condition of the bone marrow is myelofibrosis (e.g., primary myelofibrosis (PMF)), myeloid metaplasia, chronic idiopathic myelofibrosis, or primary myelofibrosis. In other embodiments, bone marrow fibrosis is associated with a hematologic disorder chosen from one or more of hairy cell leukemia, lymphoma, or multiple myeloma.

In other embodiments, the bone marrow fibrosis is associated with one or more myeloproliferative neoplasms (MPN) chosen from: essential thrombocythemia (ET), polycythemia vera (PV), mastocytosis, chronic eosinophilic leukemia, chronic neutrophilic leukemia, or other MPN.

In one embodiment, the fibrotic condition is primary myelofibrosis. Primary myelofibrosis (PMF) (also referred to in the literature as idiopathic myeloid metaplasia, and Agnogenic myeloid metaplasia) is a clonal disorder of multipotent hematopoietic progenitor cells (reviewed in Abdel-Wahab, O. et al. (2009) Annu. Rev. Med. 60:233-45; Varicchio, L. et al. (2009) Expert Rev. Hematol. 2(3):315-334; Agrawal, M. et al. (2010) Cancer 1-15). The disease is characterized by anemia, splenomegaly and extramedullary hematopoiesis, and is marked by progressive marrow fibrosis and atypical megakaryocytic hyperplasia. CD34+ stem/progenitor cells abnormally traffic in the peripheral blood and multi organ extramedullary erythropoiesis is a hallmark of the disease, especially in the spleen and liver. The bone marrow structure is altered due to progressive fibrosis, neoangiogenesis, and increased bone deposits. A significant percentage of patients with PMF have gain-of-function mutations in genes that regulate hematopoiesis, including Janus kinase 2 (JAK2) (˜50%) (e.g., JAK2^{V617F}) or the thrombopoietin receptor (MPL) (5-10%), resulting in abnormal megakaryocyte growth and differentiation. Studies have suggested that the clonal hematopoietic disorder leads to secondary proliferation of fibroblasts and excessive collagen deposition. Decreased bone marrow fibrosis can improve clinical signs and symptoms, including anemia, abnormal leukocyte counts, and splenomegaly.

Bone marrow fibrosis can be observed in several other hematologic disorders including, but not limited to hairy cell leukemia, lymphoma, and multiple myeloma. However, each of these conditions is characterized by a constellation of clinical, pathologic, and molecular findings not characteristic of PMF (see Abdel-Wahab, O. et al. (2009) supra at page 235).

In other embodiments, the bone marrow fibrosis can be secondary to non-hematologic disorders, including but not limited to, solid tumor metastases to bone marrow, autoimmune disorders (systemic lupus erythematosus, scleroderma, mixed connective tissue disorder, polymyositis), and secondary hyperparathyroidism associated with vitamin D deficiency (see Abdel-Wahab, O. et al. (2009) supra at page 235). In most cases, it is possible to distinguish between these disorders and PMF, although in rare cases the presence of the JAK2V617F or MPLW515L/K mutation can be used to demonstrate the presence of a clonal MPN and to exclude the possibility of reactive fibrosis.

The effect of hedgehog inhibitors in myelofibrosis can be characterized in mouse models available in the art as described in Varicchio, L. (2009) supra.

Monitoring a clinical improvement in a subject with bone marrow fibrosis can be evaluated by one or more of: monitoring peripheral blood counts (e.g., red blood cells, white blood cells, platelets), wherein an increase in peripheral blood counts is indicative of an improved outcome. In other embodiments, clinical improvement in a subject with bone marrow fibrosis can be evaluated by monitoring one or more of: spleen size, liver size, and size of extramedullary hematopoiesis, wherein a decrease in one or more of these parameters is indicative of an improved outcome.

In other embodiments, the fibrotic condition is a fibrotic condition of the skin. In certain embodiments, the fibrotic condition is chosen from one or more of: skin fibrosis and/or scarring, scleroderma (e.g., systemic scleroderma), or keloid.

In certain embodiments, the fibrotic condition of the gastrointestinal tract is associated with an inflammatory disorder of the gastrointestinal tract, e.g., fibrosis associated with scleroderma; radiation induced gut fibrosis; fibrosis associated with a foregut inflammatory disorder such as Barrett's esophagus and chronic gastritis, and/or fibrosis associated with a hindgut inflammatory disorder, such as inflammatory bowel disease (IBD), ulcerative colitis and Crohn's disease (see Nielsen et al., (2004) “Hedgehog Pathway Expression in Human Gut Tissues and in Inflammatory Gut Diseases” Lab. Invest. 84:1631-42). In certain embodiments, the fibrotic condition is diffuse scleroderma.

In certain embodiments, the fibrotic condition is selected from pulmonary fibrosis, bronchiectasis, interstitial lung disease; fatty liver disease; cholestatic liver disease, biliary fibrosis, hepatic fibrosis; myocardial fibrosis; and renal fibrosis.

In certain embodiments, the fibrotic condition is selected from biliary fibrosis, hepatic fibrosis, pulmonary fibrosis, myocardial fibrosis and renal fibrosis

Other fibrotic conditions that can be treated with the methods and compositions of the invention include cystic fibrosis, endomyocardial fibrosis, mediastinal fibrosis, sarcoidosis, scleroderma, spinal cord injury/fibrosis.

Evaluating the effect of hedgehog inhibitors, such as IPI-926, in several fibrotic conditions in vivo can be performed as described in Examples 2-4 below. A number of models in which fibrosis is induced are available in the art. Administration of hedgehog inhibitors, such as IPI-926, can be readily used to evaluate whether fibrosis is ameliorated in such models. Examples of such models, include but are not limited to, the unilateral ureteral obstruction model of renal fibrosis (see Chevalier et al., “Ureteral Obstruction as a Model of Renal Interstitial Fibrosis and Obstructive Nephropathy” Kidney International (2009) 75:1145-1152), the bleomycin induced model of pulmonary fibrosis (see Moore and Hogaboam “Murine Models of Pulmonary Fibrosis” Am. J. Physiol. Lung. Cell. Mol. Physiol. (2008) 294:L152-L160), a variety of liver/biliary fibrosis models (see Chuang et al., “Animal Models of Primary Biliary Cirrhosis” Clin Liver Dis (2008) 12:333-347; Omenetti, A. et al. (2007) Laboratory Investigation 87:499-514 (biliary duct-ligated model); or a number of myelofibrosis mouse models as described in Varicchio, L. (2009) supra. Regardless of the model, IPI-926 can be evaluated in essentially three paradigms: 1) test whether IPI-926 can inhibit the fibrotic state; 2) test whether IPI-926 can stop fibrotic progression once initiated; and/or 3) test whether IPI-926 can reverse the fibrotic state once initiated.

It has also been discovered that multiple fibrotic tissues up-regulate Hh ligands (see Example 1 herein). The type of Hh ligand that is up-regulated (i.e., Sonic, Indian and/or Desert) and the degree of up-regulation vary depending upon the tissue type. Thus, an aspect of the invention is a method of treating tissue fibrosis by determining whether expression of one or more hedgehog ligands is increased, then administering a hedgehog inhibitor.

In certain embodiments, the fibrotic condition is provided in a tissue (e.g., biliary tissue, liver tissue, lung tissue, heart tissue, kidney tissue, skin tissue, gut tissue) exhibiting hedgehog activity, e.g., increased hedgehog ligand expression. In certain embodiments, the tissue is biliary tissue. In certain embodiments, the tissue is liver tissue. In certain embodiments the tissue is lung tissue. In certain embodiments, the tissue is heart tissue. In certain embodiments, the tissue is kidney tissue. In certain embodiments, the tissue is skin tissue. In certain embodiments, the tissue is gut tissue. In certain embodiments, the tissue is bone marrow tissue. In certain embodiments, the tissue is epithelial tissue.

Detecting hedgehog activity in a sample or biopsied tissue can be done utilizing well-known analytical techniques, such as Reverse Transcription-Polymerase Chain Reaction analysis for mRNA encoding Gli-1 or Ptc (see Kasper et al., “Gli Transcription Factors: Mediators of Oncogenic Hedgehog Signalling” Eur. J. Cancer (2006) 42:437-445).

Ligand expression can be measured by detection of a soluble form of the ligand in peripheral blood and/or urine (e.g., by an ELISA assay or radioimmunoassay), in circulating cells (e.g., by a fluorescence-activated cell sorting (FACS) assay, an immunohistochemisty assay, or a reverse transcription polymerase chain reaction (RT-PCR) assay), or in tissue or bone marrow biopsies (e.g., by an immunohistochemistry assay, a RT-PCR assay, or by in situ hybridization). Detection of hedgehog ligand in a given patient tissue could also be assessed in vivo, by systemic administration of a labeled form of an antibody to a hedgehog ligand followed by imaging, similar to detection of PSMA in prostate cancer patients (Bander, N H Nat Clin Pract Urol 2006; 3:216-225). Expression levels in a patient can be measured at least at two time-points to determine of ligand induction has occurred. For example, hedgehog ligand expression can be measured pre- and post-therapy, pre-therapy and at one or more time-points while therapy is ongoing, or at two or more different time-points while therapy is ongoing. If a hedgehog ligand is found to be up-regulated, a hedgehog inhibitor can be administered. Thus, measurement of hedgehog ligand induction in the patient can determine whether the patient receives a hedgehog pathway inhibitor in combination with or following other therapy.

The invention also encompasses a composition for use, and use of, a hedgehog inhibitor, alone or in combination with another agent, for preparation of one or more medicaments for use in reducing fibrosis, or treatment of a fibrotic condition.

### Hedgehog Inhibitors

Suitable hedgehog inhibitors for use with the present invention include, for example, those described and disclosed in U.S. Pat. No. 7,230,004, U.S. Patent Application Publication No. 2008/0293754, U.S. Patent Application Publication No. 2008/0287420, and U.S. Patent Application Publication No. 2008/0293755, the entire disclosures of which are incorporated by reference herein.

Examples of other suitable hedgehog inhibitors include those described in U.S. Patent Application Publication Nos. US 2002/0006931, US 2007/0021493 and US 2007/0060546, and International Application Publication Nos. WO 2001/19800, WO 2001/26644, WO 2001/27135, WO 2001/49279, WO 2001/74344, WO 2003/011219, WO 2003/088970, WO 2004/020599, WO 2005/013800, WO 2005/033288, WO 2005/032343, WO 2005/042700, WO 2006/028958, WO 2006/050351, WO 2006/078283, WO 2007/054623, WO 2007/059157, WO 2007/120827, WO 2007/131201, WO 2008/070357, WO 2008/110611, WO 2008/112913, and WO 2008/131354.

Additional examples of hedgehog inhibitors include, but are not limited to, GDC-0449 (also known as RG3616 or vismodegib) described in, e.g., Von Hoff D. et al., N. Engl. J. Med. 2009; 361(12):1164-72; Robarge K. D. et al., Bioorg Med Chem. Lett. 2009; 19(19):5576-81; Yauch, R. L. et al. (2009) Science 326: 572-574; Sciencexpress: 1-3 (10.1126/science.1179386); Rudin, C. et al. (2009) New England J of Medicine 361-366 (10.1056/nejma0902903); BMS-833923 (also known as XL139) described in, e.g., in Siu L. et al., J. Clin. Oncol. 2010; 28:15s (suppl; abstr 2501); and National Institute of Health Clinical Trial Identifier No. NCT006701891; LDE-225 described, e.g., in Pan S. et al., ACS Med. Chem. Lett., 2010; 1(3): 130-134; LEQ-506 described, e.g., in National Institute of Health Clinical Trial Identifier No. NCT01106508; PF-04449913 described, e.g., in National Institute of Health Clinical Trial Identifier No. NCT00953758; Hedgehog pathway antagonists disclosed in U.S. Patent Application Publication No. 2010/0286114; SMOi2-17 described, e.g., U.S. Patent Application Publication No. 2010/0093625; SANT-1 and SANT-2 described, e.g., in Rominger C. M. et al., J. Pharmacol. Exp. Ther. 2009; 329(3):995-1005; 1-piperazinyl-4-arylphthalazines or analogues thereof, described in Lucas B. S. et al., Bioorg. Med. Chem. Lett. 2010; 20(12):3618-22.

In certain embodiments, the hedgehog inhibitor is a compound of formula (I):

or a pharmaceutically acceptable salt and/or solvate thereof;

wherein:

R¹ is H, alkyl, —OR, amino, sulfonamido, sulfamido, —OC(O)R⁵, —N(R⁵)C(O)R⁵, or a sugar;

R² is H, alkyl, alkenyl, alkynyl, aryl, cycloalkyl, nitrile, or heterocycloalkyl;

or R¹ and R² taken together form ═O, ═S, ═N(OR), ═N(R), ═N(NR₂), or ═C(R)₂;

R³ is H, alkyl, alkenyl, or alkynyl;

R⁴ is H, alkyl, alkenyl, alkynyl, aryl, cycloalkyl, heterocycloalkyl, aralkyl, heteroaryl, heteroaralkyl, haloalkyl, —OR, —C(O)R⁵, —CO₂R⁵, —SO₂R⁵, —C(O)N(R⁵)(R⁵), —[C(R)₂]_{q}—R⁵, —[(W)—N(R)C(O)]_{q}R⁵, —[(W)—C(O)]_{q}R⁵, —[(W)—C(O)]_{q}R⁵, —[(W)—OC(O)]_{q}R⁵, —[(W)—SO₂]_{q}R⁵, —[(W)—N(R⁵)SO₂]_{q}R⁵, —[(W)—C(O)N(R⁵)]_{q}R⁵, —[(W)—O]_{q}R⁵, —[(W)—N(R)]_{q}R⁵, —W—NR₃⁺X⁻ or —[(W)—S]_{q}R⁵; wherein each W is independently for each occurrence a diradical; each q is independently for each occurrence 1, 2, 3, 4, 5, or 6; and X⁻ is a halide;

each R⁵ is independently for each occurrence H, alkyl, alkenyl, alkynyl, aryl, cycloalkyl, heterocycloalkyl, aralkyl, heteroaryl, heteroaralkyl or —[C(R)₂]ₚ—R⁶; wherein p is 0-6; or any two occurrences of R⁵ on the same substituent can be taken together to form a 4-8 membered optionally substituted ring which contains 0-3 heteroatoms selected from N, O, S, and P; and

each R⁶ is independently hydroxyl, —N(R)COR, —N(R)C(O)OR, —N(R)SO₂(R), —C(O)N(R)₂, —OC(O)N(R)(R), —SO₂N(R)(R), —N(R)(R), —COOR, —C(O)N(OH)(R), —OS(O)₂OR, —S(O)₂OR, —OP(O)(OR)(OR), —NP(O)(OR)(OR), or —P(O)(OR)(OR); and

each R is independently H, alkyl, alkenyl, alkynyl, aryl, cycloalkyl or aralkyl;

provided that when R², R³ are H and R⁴ is hydroxyl; R¹ cannot be hydroxyl;

provided that when R², R³, and R⁴ are H; R¹ cannot be hydroxyl; and

provided that when R², R³, and R⁴ are H; R¹ cannot be sugar.

In certain embodiments, R¹ is H, hydroxyl, alkoxyl, aryloxy, or amino.

In some embodiments, R¹ and R² taken together along with the carbon to which they are bonded, form ═O, ═N(OR), or ═S.

In other embodiments, R³ is H and/or R⁴ is H, alkyl, hydroxyl, aralkyl, —[C(R)₂]_{q}—R⁵, —[(W)—N(R)C(O)]_{q}R⁵, —[(W)—N(R)SO₂]_{q}R⁵, —[(W)—C(O)N(R)]_{q}R⁵, —[(W)—O]_{q}R⁵, —[(W)—C(O)]_{q}R⁵, or —[(W)—C(O)]_{q}R⁵.

In yet other embodiments, R¹ is H or —OR, R² is H or alkyl, and R⁴ is H.

In yet other embodiments, R² is H or alkyl, R³ is H, alkyl, alkenyl, alkynyl, aryl, cycloalkyl, heterocycloalkyl, or aralkyl; and/or R⁴ is H, alkyl, aralkyl, —[(W)—N(R)C(O)]_{q}R⁵, —[(W)—N(R)SO₂]_{q}R⁵, —[(W)—C(O)N(R)]_{q}R⁵, —[(W)—O]_{q}R⁵, —[(W)—C(O)]_{q}R⁵, or —[(W)—C(O)O]_{q}R⁵.

In yet other embodiments, R¹ is sulfonamide.

Specific examples of hedgehog inhibitors include compounds, or pharmaceutically acceptable salts and/or solvates thereof, described in U.S. Patent Application 2008/0293754 and also provided below in Table 1:

```
TABLE 1
  1
  2
  3
  4
  5
  6
  7
  8
  9
10
11
12
13
14
15
16
17
18
19
20
21
22
23
24
25
26
27
28
29
30
31
32
33
34
35
36
37
38
39
40
41
42
43
44
45
46
47
48
```

Other examples of hedgehog inhibitors include compounds, or pharmaceutically acceptable salts and/or solvates thereof, described in U.S. Pat. No. 7,230,004 and also provided below in Table 2:

TABLE 2
49
50
51
52
53
54
55
56
57
58
59
60
61
62
63
64
65
66
67
68
69
70
71
72
73
74
75
76
77
78
79
80
81
82
83
84
85
86
87
88
89
90
91
92
93
94
95
96
97
98
99
100
101
102
103

Yet other examples of hedgehog inhibitors include compounds, or pharmaceutically acceptable salts and/or solvates thereof, described in U.S. Patent Application No. 2008/0287420, and also provided below in Table 3:

TABLE 3
104
105
106
107
108
109
110
111
112
113
114
115
116
117
118

Still yet other examples of hedgehog inhibitors include compounds, or pharmaceutically acceptable salts and/or solvates thereof, described in U.S. Patent Application No. 2008/0293755, and also provided below in Table 4:

TABLE 4
119
120
121
122
123
124
125
126
127
128
129
130
131
132
133
134
135
136
137

In certain embodiments, the hedgehog inhibitor is the compound of the formula 32 (i.e., IPI-926):

or a pharmaceutically acceptable salt and/or solvate thereof.

In certain embodiments, the pharmaceutically acceptable salt of compound 32 is the hydrochloric, hydrobromic, phosphoric, sulfuric, nitric, perchloric, adipic, alginic, ascorbic, aspartic, 2-acetoxybenzoic, benzenesulfonic, benzoic, bisulfonic, boric, butyric, camphoric, camphorsulfonic, citric, cyclopentanepropionic, digluconic, dodecylsulfonic, ethanesulfonic, 1,2-ethanedisulfonic, formic, fumaric, glucoheptonic, glycerophosphonic, gluconic, hemisulfonic, heptanoic, hexanoic, hydroiodic, 2-hydroxyethanesulfonic, hydroxymaleic, isothionic, lactobionic, lactic, lauric, lauryl sulfonic, malic, maleic, malonic, methanesulfonic, 2-naphthalenesulfonic, napthylic, nicotinic, oleic, oxalic, palmitic, pamoic, pectinic, persulfonic, 3-phenylpropionic, picric, pivalic, propionic, phenylacetic, stearic, succinic, salicyclic, sulfanilic, tartaric, thiocyanic, p-toluenesulfonic, undecanoic or valeric acid addition salt.

In certain embodiments, the pharmaceutically acceptable salt of compound 32 is the hydrochloric acid addition salt.

In certain embodiments, the hedgehog inhibitor is an isopropanol (IPA) solvate of compound 32 or a pharmaceutically acceptable salt thereof.

In certain embodiments, the hedgehog inhibitor is the compound of formula 32 or a pharmaceutically acceptable salt and/or solvate thereof and the fibrotic condition is a fibrotic condition of the lung, a fibrotic condition of the liver, a fibrotic condition of the hear or vasculaturet, a fibrotic condition of the kidney, a fibrotic condition of the skin, a fibrotic condition of the gastrointestinal tract; a fibrotic condition of the bone marrow, or a combination thereof.

In certain embodiments, the hedgehog inhibitor is the compound of formula 32 or a pharmaceutically acceptable salt and/or solvate thereof and the fibrotic condition is a fibrotic condition of the lung. In certain embodiments, the hedgehog inhibitor is the compound of formula 32 or a pharmaceutically acceptable salt and/or solvate thereof and the fibrotic condition is pulmonary fibrosis. In certain embodiments, the pulmonary fibrosis is idiopathic pulmonary fibrosis (IPF) or usual interstitial pneumonitis (UIP). In certain embodiments, the hedgehog inhibitor is the compound of formula 32 or a pharmaceutically acceptable salt and/or solvate thereof and the fibrotic condition is interstitial lung disease. In certain embodiments, the interstitial lung disease is cryptogenic fibrosing alveolitis (CFA). In certain embodiments, the hedgehog inhibitor is the compound of formula 32 or a pharmaceutically acceptable salt and/or solvate thereof and the fibrotic condition is bronchiectasis.

In certain embodiments, the hedgehog inhibitor is the compound of formula 32 or a pharmaceutically acceptable salt and/or solvate thereof and the fibrotic condition is a fibrotic condition of the liver. In certain embodiments, the hedgehog inhibitor is the compound of formula 32 or a pharmaceutically acceptable salt and/or solvate thereof and the fibrotic condition is fatty liver disease. In certain embodiments, the fatty liver disease is steatosis. In certain embodiments, the steatosis is nonalcoholic steatohepatitis (NASH). In certain embodiments, the hedgehog inhibitor is the compound of formula 32 or a pharmaceutically acceptable salt and/or solvate thereof and the fibrotic condition is cholestatic liver disease. In certain embodiments, the cholestatic liver disease is primary biliary cirrhosis (PBC). In certain embodiments, the hedgehog inhibitor is the compound of formula 32 or a pharmaceutically acceptable salt and/or solvate thereof and the fibrotic condition is biliary fibrosis. In certain embodiments, the hedgehog inhibitor is the compound of formula 32 or a pharmaceutically acceptable salt and/or solvate thereof and the fibrotic condition is hepatic fibrosis.

In certain embodiments, the hedgehog inhibitor is the compound of formula 32 or a pharmaceutically acceptable salt and/or solvate thereof and the fibrotic condition is a fibrotic condition of the heart. In certain embodiments, the hedgehog inhibitor is the compound of formula 32 or a pharmaceutically acceptable salt and/or solvate thereof and the fibrotic condition is myocardial fibrosis.

In certain embodiments, the hedgehog inhibitor is the compound of formula 32 or a pharmaceutically acceptable salt and/or solvate thereof and the fibrotic condition is a fibrotic condition of the kidney. In certain embodiments, the hedgehog inhibitor is the compound of formula 32 or a pharmaceutically acceptable salt and/or solvate thereof and the fibrotic condition is renal fibrosis.

In certain embodiments, the hedgehog inhibitor is the compound of formula 32 or a pharmaceutically acceptable salt and/or solvate thereof and the fibrotic condition is a fibrotic condition of the skin. In certain embodiments, the hedgehog inhibitor is the compound of formula 32 or a pharmaceutically acceptable salt and/or solvate thereof and the fibrotic condition is skin fibrosis (e.g., scarring). In certain embodiments, the hedgehog inhibitor is the compound of formula 32 or a pharmaceutically acceptable salt and/or solvate thereof and the fibrotic condition is scleroderma. In certain embodiments, the scleroderma is systemic sclerderma. In certain embodiments, the hedgehog inhibitor is the compound of formula 32 or a pharmaceutically acceptable salt and/or solvate thereof and the fibrotic condition is nephrogenic systemic fibrosis. In certain embodiments, the hedgehog inhibitor is the compound of formula 32 or a pharmaceutically acceptable salt and/or solvate thereof and the fibrotic condition is keloid.

In certain embodiments, the hedgehog inhibitor is the compound of formula 32 or a pharmaceutically acceptable salt and/or solvate thereof and the fibrotic condition is a fibrotic condition of the gastrointestinal tract. In certain embodiments, the hedgehog inhibitor is the compound of formula 32 or a pharmaceutically acceptable salt and/or solvate thereof and the fibrotic condition is diffuse scleroderma. In certain embodiments, the hedgehog inhibitor is the compound of formula 32 or a pharmaceutically acceptable salt and/or solvate thereof and the fibrotic condition is Crohn's disease.

In certain embodiments, the hedgehog inhibitor is the compound of formula 32 or a pharmaceutically acceptable salt and/or solvate thereof and the fibrotic condition is selected from pulmonary fibrosis, bronchiectasis, interstitial lung disease; fatty liver disease; cholestatic liver disease, biliary fibrosis, hepatic fibrosis; myocardial fibrosis; and renal fibrosis. In certain embodiments, the hedgehog inhibitor is the compound of formula 32 or a pharmaceutically acceptable salt and/or solvate thereof and the fibrotic condition is selected from biliary fibrosis, hepatic fibrosis, pulmonary fibrosis, myocardial fibrosis and renal fibrosis. In certain embodiments, the hedgehog inhibitor is the compound of formula 32 or a pharmaceutically acceptable salt and/or solvate thereof and the fibrotic condition is selected from biliary fibrosis, hepatic fibrosis, pulmonary fibrosis and renal fibrosis. In certain embodiments, the hedgehog inhibitor is the compound of formula 32 or a pharmaceutically acceptable salt and/or solvate thereof and the fibrotic condition is selected from biliary fibrosis, hepatic fibrosis and pulmonary fibrosis. In certain embodiments, the hedgehog inhibitor is the compound of formula 32 or a pharmaceutically acceptable salt and/or solvate thereof and the fibrotic condition is selected from biliary fibrosis and hepatic fibrosis.

### Pharmaceutical Compositions, Dosage and Administration

In some embodiments, the above-described method comprises providing the hedgehog inhibitor in a pharmaceutical composition.

Pharmaceutical compositions can be formulated for administration in solid or liquid form, including those adapted for the following: oral administration, for example, drenches (e.g., aqueous or non-aqueous solutions or suspensions), tablets (e.g., those targeted for buccal, sublingual, and systemic absorption), capsules, boluses, powders, granules, pastes for application to the tongue; parenteral administration, for example, by subcutaneous, intramuscular, intravenous or epidural injection such as, for example, a sterile solution or suspension, or sustained-release formulation; topical application, for example, as a cream, ointment, or a controlled-release patch or spray applied to the skin; intravaginally or intrarectally, for example, as a pessary, cream or foam; sublingually; ocularly; transdermally; pulmonarily; or nasally.

Pharmaceutically acceptable excipients include any and all fillers, binders, surfactants, disintegrants, sugars, polymers, antioxidants, solubilizing or suspending agents, chelating agents, preservatives, buffering agents and/or lubricating agents, or combinations thereof, as suited to the particular dosage form desired and according to the judgment of the formulator. Remington's Pharmaceutical Sciences, Sixteenth Edition, E. W. Martin (Mack Publishing Co., Easton, Pa., 1980) discloses various pharmaceutically acceptable excipients used in preparing compositions and known techniques for the preparation thereof. Except insofar as any conventional carrier medium is incompatible with the compounds disclosed herein, such as by producing any undesirable biological effect or otherwise interacting in a deleterious manner with any component of the composition, its use is contemplated to be within the scope of this invention. In general, the compositions are prepared by uniformly and intimately bringing into association the hedgehog inhibitor with one or more excipients and then, if necessary, shaping the product.

When the hedgehog inhibitor is administered to humans or animals it can be given per se or as a pharmaceutical composition containing, for example, about 0.1 to 99%, or about 10 to 50%, or about 10 to 40%, or about 10 to 30%, or about 10 to 20%, or about 10 to 15% of the hedgehog inhibitor in combination with a pharmaceutically acceptable excipient.

Actual dosage levels of the hedgehog inhibitor in the pharmaceutical compositions can be varied so as to obtain an amount of the active ingredient which is effective to achieve the desired therapeutic response for a particular subject, composition, and mode of administration, without being toxic to the subject.

The selected dosage level will depend upon a variety of factors including, for example, the activity of the particular hedgehog inhibitor employed, the route of administration, the time of administration, the rate of excretion or metabolism, the rate and extent of absorption, the duration of the treatment, other drugs, compounds or materials used in combination with the hedgehog inhibitor, the age, sex, weight, condition, general health and prior medical history of the subject, and other similar factors well known in the medical arts.

In general, a suitable daily dose of a hedgehog inhibitor will be that amount which is the lowest dose effective to produce a therapeutic effect. Such an effective dose will generally depend upon the factors described above. Generally, oral, intravenous and subcutaneous doses of the hedgehog inhibitor for a subject, when used for the indicated effects, will range from about 0.0001 mg to about 100 mg per day, or about 0.001 mg to about 100 mg per day, or about 0.01 mg to about 100 mg per day, or about 0.1 mg to about 100 mg per day, or about 0.0001 mg to about 500 mg per day, or about 0.001 mg to about 500 mg per day, or about 0.01 mg to about 500 mg per day, or about 0.1 mg to about 500 mg per day.

The subject receiving the treatment can be any animal in need, including primates (e.g. humans), equines, cattle, swine, sheep, poultry, dogs, cats, mice and rats.

The hedgehog inhibitor can be administered daily, every other day, three times a week, twice a week, weekly, or bi-weekly. The dosing schedule can include a “drug holiday,” i.e., the drug can be administered for two weeks on, one week off, or three weeks on, one week off, or four weeks on, one week off, etc., or continuously, without a drug holiday. The hedgehog inhibitor can be administered orally, intravenously, intraperitoneally, topically, transdermally, intramuscularly, subcutaneously, intranasally, sublingually, or by any other route.

### Combination Therapies

The hedgehog inhibitor can be administered in combination with one or more therapeutic agents. Exemplary therapeutic agents include, but are not limited to, anti-fibrotics, corticosteroids, anti-inflammatories, immunosuppressants, chemotherapeutic agents, anti-metabolites, and immunomodulators.

By “in combination with,” it is not intended to imply that the therapeutic agent and the hedgehog inhibitor must be administered at the same time and/or formulated for delivery together, although these methods of delivery are within the scope of the invention. The hedgehog inhibitor can be administered concurrently with, prior to, or subsequent to, one or more other additional agents. In general, each therapeutic agent will be administered at a dose and/or on a time schedule determined for that particular agent. In will further be appreciated that the therapeutic agent utilized in this combination can be administered together in a single composition or administered separately in different compositions. The particular combination to employ in a regimen will take into account compatibility of the hedgehog inhibitor with the agent and/or the desired therapeutic effect to be achieved.

In general, it is expected that additional therapeutic agents employed in combination be utilized at levels that do not exceed the levels at which they are utilized individually. In some embodiments, the levels utilized in combination will be lower than those utilized individually. The determination of the mode of administration and the correct dosage for each agent or combination therapy is well within the knowledge of the skilled clinician.

In some embodiments, the hedgehog inhibitor is a first line treatment for the fibrotic condition, i.e., it is used in a subject who has not been previously administered another drug intended to treat the condition.

In other embodiments, the hedgehog inhibitor is a second line treatment for the fibrotic condition, i.e., it is used in a subject who has been previously administered another drug intended to treat the condition.

In other embodiments, the hedgehog inhibitor is a third or fourth line treatment for the fibrotic condition, i.e., it is used in a subject who has been previously administered two or three other drugs intended to treat the condition.

In some embodiments, a hedgehog inhibitor is administered to a subject following surgical excision/removal.

In some embodiments, a hedgehog inhibitor is administered to a subject before, during, and/or after radiation treatment.

In embodiments where two agents are administered, the two agents can be administered concurrently (i.e., essentially at the same time, or within the same treatment) or sequentially (i.e., one immediately following the other, or alternatively, with a gap in between administration of the two). In some embodiments, the hedgehog inhibitor is administered sequentially (i.e., after the first therapeutic).

In embodiments where a fibrotic condition of the bone marrow is treated, the hedgehog inhibitor can be administered in combination with an agent chosen from a Jak2 inhibitor (including, but not limited to, INCB018424, XL019, TG101348, or TG101209), an immunomodulator, e.g., an IMID (including, but not limited to thalidomide, lenalidomide, or panolinomide), hydroxyurea, an androgen, erythropoietic stimulating agents, prednisone, danazol, HDAC inhibitors, or other agents or therapeutic modalities (e.g., stem cell transplants, or radiation).

An example of suitable therapeutics for use in combination with the hedgehog inhibitor for treatment of heart fibrosis includes, but is not limited to, eplerenone, furosemide, pycnogenol, spironolactone, TcNC100692, torasemide (e.g., prolonged release form of torasemide), and combinations thereof.

An example of suitable therapeutics for use in combination with the hedgehog inhibitor for treatment of kidney fibrosis includes, but is not limited to, cyclosporine, cyclosporine A, daclizumab, everolimus, gadofoveset trisodium (ABLAVAR®), imatinib mesylate (GLEEVEC®), matinib mesylate, methotrexate, mycophenolate mofetil, prednisone, sirolimus, spironolactone, STX-100, tamoxifen, TheraCLEC™, and combinations thereof.

An example of suitable therapeutics for use in combination with the hedgehog inhibitor for treatment of skin fibrosis includes, but is not limited to, Bosentan (Tracleer), p144, pentoxifylline; pirfenidone; pravastatin, STI571, Vitamin E, and combinations thereof.

An example of suitable therapeutics for use in combination with the hedgehog inhibitor for treatment of gastrointestinal fibrosis includes, but is not limited to, ALTU-135, bucelipase alfa (INN), DCI1020, EUR-1008 (ZENPEP™), ibuprofen, Lym-X-Sorb powder, pancrease MT, pancrelipase (e.g., pancrelipase delayed release), pentade canoic acid (PA), repaglinide, TheraCLEC™, triheptadecanoin (THA), ULTRASE MT20, ursodiol, and combinations thereof.

An example of suitable therapeutics for use in combination with the hedgehog inhibitor for treatment of lung fibrosis includes, but is not limited to, 18-FDG, AB0024, ACT-064992 (macitentan), aerosol interferon-gamma, aerosolized human plasma-derived alpha-1 antitrypsin, alpha1-proteinase inhibitor, ambrisentan, amikacin, amiloride, amitriptyline, anti-pseudomonas IgY gargle, ARIKACE™, AUREXIS® (tefibazumab), AZAPRED, azathioprine, azithromycin, azithromycin, AZLI, aztreonam lysine, BIBF1120, Bio-25 probiotic, bosentan, Bramitob®, calfactant aerosol, captopril, CC-930, ceftazidime, ceftazidime, cholecalciferol (Vitamin D3), ciprofloxacin (CIPRO®, BAYQ3939), CNTO 888, colistin CF, combined Plasma Exchange (PEX), rituximab, and corticosteroids, cyclophosphamide, dapsone, dasatinib, denufosol tetrasodium (INS37217), dornase alfa (PULMOZYME®), EPI-hNE4, erythromycin, etanercept, FG-3019, fluticasone, FTI, GC1008, GS-9411, hypertonic saline, ibuprofen, iloprost inhalation, imatinib mesylate (GLEEVEC®), inhaled sodium bicarbonate, inhaled sodium pyruvate, interferon gamma-1b, interferon-alpha lozenges, isotonic saline, IW001, KB001, losartan, lucinactant, mannitol, meropenem, meropenem infusion, miglustat, minocycline, Moli1901, MP-376 (levofloxacin solution for inhalation), mucoid exopolysaccharide P. aeruginosa immune globulin IV, mycophenolate mofetil, n-acetylcysteine, N-acetylcysteine (NAC), NaCl 6%, nitric oxide for inhalation, obramycin, octreotide, oligoG CF-5/20, Omalizumab, pioglitazone, piperacillin-tazobactam, pirfenidone, pomalidomide (CC-4047), prednisone, prevastatin, PRM-151, QAX576, rhDNAse, SB656933, SB-656933-AAA, sildenafil, tamoxifen, technetium [Tc-99 m] sulfur colloid and Indium [In-111] DTPA, tetrathiomolybdate, thalidomide, ticarcillin-clavulanate, tiotropium bromide, tiotropium RESPIMAT® inhaler, tobramycin (GERNEBCIN®), treprostinil, uridine, valganciclovir (VALCYTE®), vardenafil, vitamin D3, xylitol, zileuton, and combinations thereof.

An example of suitable therapeutics for use in combination with the hedgehog inhibitor for treatment of liver fibrosis includes, but is not limited to, adefovir dipivoxil, candesartan, colchicine, combined ATG, mycophenolate mofetil, and tacrolimus, combined cyclosporine microemulsion and tacrolimus, elastometry, everolimus, FG-3019, Fuzheng Huayu, GI262570, glycyrrhizin (monoammonium glycyrrhizinate, glycine, L-cysteine monohydrochloride), interferon gamma-1b, irbesartan, losartan, oltipraz, ORAL IMPACT®, peginterferon alfa-2a, combined peginterferon alfa-2a and ribavirin, peginterferon alfa-2b (SCH 54031), combined peginterferon alpha-2b and ribavirin, praziquantel, prazosin, raltegravir, ribavirin (REBETOL®, SCH 18908), ritonavir-boosted protease inhibitor, pentoxyphilline, tacrolimus, tauroursodeoxycholic acid, tocopherol, ursodiol, warfarin, and combinations thereof.

An example of other suitable therapeutics for use in combination with the hedgehog inhibitor for treatment of cystic fibrosis includes, but is not limited to, 552-02, 5-methyltetrahydrofolate and vitamin B12, Ad5-CB-CFTR, Adeno-associated virus-CFTR vector, albuterol, alendronate, alpha tocopherol plus ascorbic acid, amiloride HCl, aquADEK™, ataluren (PTC124), AZD1236, AZD9668, azithromycin, bevacizumab, biaxin (clarithromycin), BIIL 283 BS (amelubent), buprofen, calcium carbonate, ceftazidime, cholecalciferol, choline supplementation, CPX, cystic fibrosis transmembrane conductance regulator, DHA-rich supplement, digitoxin, cocosahexaenoic acid (DHA), doxycycline, ECGC, ecombinant human IGF-1, educed glutathione sodium salt, ergocalciferol (vitamin D2), fluorometholone, gadobutrol (GADOVIST®, BAY86-4875), gentamicin, ghrelin, glargine, glutamine, growth hormone, GS-9411, H5.001CBCFTR, human recombinant growth hormone, hydroxychloroquine, hyperbaric oxygen, hypertonic saline, IH636 grape seed proanthocyanidin extract, insulin, interferon gamma-1b, IoGen (molecular iodine), iosartan potassium, isotonic saline, itraconazole, IV gallium nitrate (GANITE®) infusion, ketorolac acetate, lansoprazole, L-arginine, linezolid, lubiprostone, meropenem, miglustat, MP-376 (levofloxacin solution for inhalation), normal saline IV, Nutropin AQ, omega-3 triglycerides, pGM169/GL67A, pGT-1 gene lipid complex, pioglitazone, PTC124, QAU145, salmeterol, SB656933, SB656933, simvastatin, sitagliptin, sodium 4-phenylbutyrate, standardized turmeric root extract, tgAAVCF, TNF blocker, TOBI, tobramycin, tocotrienol, unconjugated Isoflavones 100, vitamin: choline bitartrate (2-hydroxyethyl) trimethylammonium salt 1:1, VX-770, VX-809, Zinc acetate, and combinations thereof.

### EXAMPLES

The invention now being generally described, it will be more readily understood by reference to the following examples, which are included merely for purposes of illustration of certain aspects and embodiments of the present invention, and are not intended to limit the invention.

### Example 1A

### IPI-926 and the Treatment of Fibrotic Conditions

### Materials and Methods

Tissues were harvested and fixed in 10% Neutral Buffered Formalin (Richard-Allen) for 24-48 Hr, embedded in paraffin, sectioned at 5 μm thick, and stained with hematoxylin-eosin (H&E).

Immunohistochemical staining for the hSHH using the polymer system was performed by rabbit monoclonal antibody [hSHH (clone EP1190Y), Abcam Inc., Cambridge, Mass., U.S.A.]. Formalin fixed paraffin-embedded tissue sections were deparaffinized by routine techniques. The slides were incubated with Sodium Citrate Buffer, pH 6, in the pressure cooker for 20 min and then cooled for 20 min for the epitope retrieval. The endogenous peroxidase activity was blocked with 3% hydrogen peroxide in deionized water for 10 min at room temperature. Sections were incubated with Background Sniper (Biocare Medical), fetal calf serum equivalent, for 20 min at room temperature to block non-specific binding, and then the primary antibody at a dilution 1:800 in DaVinci Green Antibody Diluent (Biocare Medical) for 90 min at room temperature. Sections were washed with TBS and incubated with Rabbit-on-Rodent Polymer (Biocare Medical) for 45 min at room temperature. After washing in TBS, the sections were stained with 3.3 diaminobenzidine tetrahydrochloride (DAKO) as chromogen for 5 min for immunohistochemical demonstration of SHH. Finally, the sections were counter-stained with CAT hematoxylin (Biocare Medical) and mounted in Permount.

The Hedgehog pathway is a ligand/receptor system in which ligand (Sonic, Indian or Desert hedgehog protein), upon binding to its receptor elicits a biochemical signal generated by the protein Smoothened resulting in, among other things, the upregulation of the mRNA for the gene Gli1. A number of tissues have been identified, such as cardiac muscle, kidney and liver, in which SHH ligand appears to be expressed in a diseased state involving fibrosis.

More specifically, histological tissue cross sections of biopsies of diseased tissues from human heart, kidney and liver were immunohistochemically stained with rabbit monoclonal antibody to Sonic Hedgehog (anti-hSHH, clone EP1190Y, described above). Tissues with positive antibody binding to hSHH appeared dark brown (data not shown). Each of these immunohistochemical stained tissues is described as follows:

A biopsy of diseased human heart tissue stained with an anti-hSHH (10× objective) antibody showed strong positive staining of he cardiac muscles show to hSHH (data not shown).

A biopsy of diseased human kidney tissue stained with an anti-hSHH antibody (10× objective) showed positive, strong nuclear staining to hSHH in the epithelia of the neoplastic papillary lesions show, while the fibrotic dysplastic glomeruli adjacent to the lesions show light staining (data not shown).

A biopsy of diseased human liver tissue showed positive staining to hSHH localized adjacent to fibrotic dysplasia (4× objective) (data not shown). The patient had high inflammatory content with marked edema and bile salt deposition.

Given the immunohistochemical staining results, Applicants concluded that SHH ligand produced by parenchymal cells of an organ, such as kidney, liver or cardiac muscle, stimulate fibrosis in the stromal cells of that same organ. A number of models in which fibrosis is induced are available in the art. Administration of hedgehog inhibitors, such as IPI-926, can be used to evaluate whether fibrosis is ameliorated in such models. Examples of such models, include but are not limited to, the unilateral ureteral obstruction model of renal fibrosis (see Chevalier et al., “Ureteral Obstruction as a Model of Renal Interstitial Fibrosis and Obstructive Nephropathy” Kidney International (2009) 75:1145-1152), the bleomycin induced model of pulmonary fibrosis (see Moore and Hogaboam “Murine Models of Pulmonary Fibrosis” Am. J. Physiol. Lung. Cell. Mol. Physiol. (2008) 294:L152-L160), or a variety of liver/biliary fibrosis models (see Chuang et al., “Animal Models of Primary Biliary Cirrhosis” Clin Liver Dis (2008) 12:333-347). Regardless of the model, IPI-926 can be evaluated in essentially three paradigms: 1) test whether IPI-926 can inhibit the fibrotic state; 2) test whether IPI-926 can stop fibrotic progression once initiated; and/or 3) test whether IPI-926 can reverse the fibrotic state once initiated.

### Example 1B

### Monitoring Hedgehog Signaling in Human and Murine Tissues

Additional protocols for evaluating hedgehog signaling are as follows:

I. IHC protocol to detect Gli1 on murine and human tissues:
Rabbit anti-mouse Gli1 (Novus Biologicals NBP1-03294), Whole Rabit IgG2
Positive control: RMS, BCC

### Secondary: Rabbit-on-Rodent Polymer (Biocare Medical RMR622)

Background Sniper (Biocare Medical BS966) or Rodent Block (Biocare medical)
Wash buffer: TBS+0.5% Tween
Citrate Retrieval buffer
DaVinci Green Primary Ab diluent (Biocrae Medical PD900)
DAB Enhancer (Zymed Cat. No. 00-2021)

### A. Dewaxing Paraffin-embedded Slides

1. 3×, 3 mins Xylene.
2. 1×, 3 mins 100% EtOH.
3. 1×, 3 mins 95% EtOH.
4. 1×, 3 mins 70% EtOH.
5. 1×, 3 mins dH2O.

### B. Unmasking/Chamber set up

1. Transfer slides to the staining jar and add Citrate target retrieval buffer to cover slides.
2. Set pressure cooker to steam for 40 minutes. Press start.
3. Once water is boiling, place the lid on and keep under pressure for 20 mins.
4. Remove staining jar and cool for 20 mins at room temp.
5. Draw the borders with the PAP pen.
6. Add PBS to keep the slides from drying.

### C. Block Endogenous Peroxidase

1. Add 3% H2O2/H2O 2× for 5 min (rinse with dH2O in between).
2. Rinse 1× with dH2O.
   D. Block any background with Background Sniper on human tissue or with Rodent Block M on murine tissues for 1 hr at RT.

### E. Primary Ab

1. Dilute the Gli1, 1:200, in DaVinci Green Diluent. Add ratIgG2a, at same dilution, to the negative controls. Add a volume of 300 μl to each slide.
2. Incubate for 1.5 hours at room temperature.

### F. Secondary Ab/ABC/DAB

1. Rinse 3× for 5 mins with wash buffer.
2. Secondary Ab: Add 300 μl of polymer directly to the slide for 45 mins
3. Rinse 3× for 5 mins with wash buffer.

### G. DAB/Developing Slides

1. Prepare the DAB solution by adding 1 drop of the chromogen to 1.0 ml of the DAB substrate, provided in the kit.

2. Add the DAB to chambers.

3. Develop the slides for 5 minutes. Add dH2O to stop the reaction.

4. Remove the slides from the chambers and proceed to counterstaining.

### H. DAB Enhancer (Zymed)

1. Apply DAB enhancer as per manufacturer's instructions

I. Counter stain: Place the slides in the staining rack and jar.

I. Wash in dH20 1× 5 min

2. Wash 45 secs in Cat Hematoxylin (Biocare medical)

3. Wash 1 min in dH2O

4. Wash 45 secs in Scott's Tap Water Substitute

6. Wash 1 min in dH2O

7. Wash 2× for 1 min in 95% EtOH

8. Wash 2× for 3 min in 100% EtOH

9. Wash 3× for 3 min each in Xylene.

Coverslip and let dry for 24 hours

### II. IHC Protocol for Detecting Staining Using Rabbit Anti-Human Sonic Hedgehog mAb

Rabbit anti-human SHh (Abcam# ab53281). Isotype control: Whole Rabbit IgG
Positive control: Pancreatic xenograft
Secondary: Rabbit-on-Rodent Polymer (Biocare Medical RMR622) for xenografts or Mach 2
(Biocare Medical RHRP520) for human tissue
Wash buffer: PBS+0.5% Tween
Citrate Retrieval buffer
DaVinci Green Primary Ab diluent (Biocrae Medical PD900)
DAB Enhancer (Zymed Cat. No. 00-2021)

### A. Dewaxing Paraffin-Embedded Slides

6. 3×, 3 mins Xylene.

7. 1×, 3 mins 100% EtOH.

8. 1×, 3 mins 95% EtOH.

9. 1×, 3 mins 70% EtOH.

10. 1×, 3 mins dH2O.

11. 1×, 5 mins PBS

### B. Unmasking/Chamber set up

1. Transfer slides to the staining jar and add just enough Citrate target retrieval buffer to cover slides.

2. Set pressure cooker to steam for 40 minutes. Press start.

3. Once water is boiling, place the lid on and keep under pressure for 20 mins.

4. Remove staining jar and let stand for 10 mins, then cool for 30 mins at room temp.

5. Draw the borders with the PAP pen.

6. Add PBS to keep the slides from drying.

### C. Block Endogenous Peroxidase

1. Add 3% H2O2/H2O 2× for 5 min (rinse with dH2O in between).

2. Rinse 1× with dH2O.

### E. Primary Ab

1. Dilute the SHH, 1:2000 (optimized on the DAKO autostainer), in DaVinci Green Diluent. Add RabbitIgG, at same dilution, to the negative controls. Add a volume of 300 μl to each slide.
2. Incubate for 90 minutes at room temperature.

### F. Secondary Ab/ABC/DAB

1. Rinse 3× for 5 mins with wash buffer.

2. Secondary Ab: Add 300 μl of polymer directly to the slide for 45 mins.

3. Rinse 3× for 5 mins with wash buffer.

### G. DAB/Developing Slides

1. Prepare the DAB solution by adding 1 drop of the chromogen to 1.0 ml of the DAB substrate, provided in the kit. Mix well and keep away from light by covering the tube with aluminum foil.

2. In the dark, add the DAB to chambers. Make sure the volume added to each chamber is held constant—300 μl.

3. Develop the slides for 5 minutes. Quickly add dH2O to each chamber to stop the reaction.

4. Remove the slides from the chambers and proceed to counterstaining.

### H. DAB Enhancer (Zymed)

1. Apply DAB enhancer as per manufacturer's instructions

### I. Counter Stain: Place the Slides in the Staining Rack and Jar.

1. Wash in dH20 1× 5 min

2. Wash 45 secs in 1:5 Gill's III Hematoxylin or 35-45 secs in Cat Hematoxylin

3. Wash 1 min in dH2O

4. Wash 45 secs in Scott's Tap Water Substitute

6. Wash 1 min in dH2O

7. Wash 2× for 1 min in 95% EtOH

8. Wash 2× for 3 min in 100% EtOH

9. Wash 3× for 3 min each in Xylene.

Coverslip and let dry for 24 hours

### Example 2A

### Efficacy of IPI 926 on the Severity of Pulmonary Fibrosis Induced by Bleomycin in C57BL/6 Mice

This Example evaluates the efficacy of different dosing schedules of IPI 926 (a Smoothened inhibitor) on the severity of pulmonary fibrosis induced by bleomycin.

### 2.1 Summary of Methods

Thirty-two C57/B6 mice were prospectively randomized into four equally sized groups of eight animals each. An additional four mice served as naïve controls. Pulmonary fibrosis was induced with a single dose of bleomycin (4 U/kg) given intra-nasally on Day 0. Animals' activity and weight were evaluated daily. IPI 926 or vehicle was given every other day from 10 days prior to bleomycin until sacrifice on Day 30, or IPI926 was given beginning on either Day 0 or Day 14 on alternate days until the end of the study. At the end of the study on Day 30, all mice were euthanized and bronchiolar lavage performed to enable an evaluation of the inflammatory infiltrate in the lungs and collect the fluid for possible cytokine evaluation. One lung from each animal was collected for histology, while the other was snap frozen and retained for possible additional analyses.

### 2.2 Results

In the control group treated with bleomycin and vehicle, a small reduction was seen in weight gain, but this was not statistically significant, total white cell counts were unchanged, while differential counts showed a slight shift to neutrophils. Lung histology in this group showed significant chronic inflammation and interstitial fibrosis when compared to the naïve controls. Mice treated with IPI926 showed reductions in weight gain relative to vehicle controls and reductions in the number of white cells in the bronchiolar lavage. IPI926 was effective in reducing the histological features of bleomycin induced pulmonary fibrosis and the reductions observed were more pronounced in groups that had been dosed for a longer period. However the reductions observed were not statistically significant when compared to the vehicle controls.

### 2.3 Summary of Results

1. Significant reductions in weight gain were seen in animals treated with IPI926 on alternating days from Day −10 to Day 30, or from Day 0 to Day 30 relative to vehicle controls. In addition, 2 mice in the group treated with IPI926 from Day −10 to Day 30 died on Days 13 and 16.

2. Evaluation of the bronchiolar lavage fluids for cellularity revealed no substantial differences between the naïve animals and the control group treated with bleomycin and vehicle. Reductions in white cell count were observed in all groups treated with IPI926, with the greatest reductions being seen in the group treated from Day 14 to Day 30.

3. Under the conditions of the study, treatment of a bleomycin-induced pulmonary fibrosis model in mice with IPI-926 at 40 mg/kg results in a reduction in pulmonary fibrosis and associated inflammation compared to the vehicle control group. The dosing regimen from Day-10 to Day 30 shows the greatest effect in reducing pulmonary fibrosis in the model. None of the treatment effects is statistically significant compared to the vehicle control.

### 2.4 Background

Pulmonary fibrosis is the formation of scar tissue within the lungs, which results in a loss of function. The consequences of this are shortness of breath, which progresses to discomfort in the chest weakness and fatigue, and ultimately to loss of appetite and rapid weight-loss. Approximately 500,000 people in the US and 5 million worldwide suffer from pulmonary fibrosis, and 40,000 people in the US die annually from the disease. Pulmonary fibrosis has a number of causes, including radiation therapy, but can also be due to smoking or hereditary factors (Meltzer, E B et al. (2008) Orphanet J. Rare Dis. 3:8).

Pulmonary fibrosis frequently occurs as a secondary effect in disease processes such as asbestosis and silicosis, and is known to be more prevalent in certain occupations such as coal miner, ship workers and sand blasters where exposure to environmental pollutants is an occupational hazard (Green, F H et al. (2007) Toxicol Pathol. 35:136-47). Other factors that contribute to pulmonary fibrosis include cigarette smoking, and autoimmune connective tissue disorders, like rheumatoid arthritis, scleroderma and systemic lupus erythematosus (SLE) (Leslie, K O et al. (2007) Semin Respir Crit. Care Med. 28:369-78; Swigris, J J et al. (2008) Chest. 133:271-80; and Antoniou, K M et al. (2008) Curr Opin Rheumatol. 20:686-91). Other connective tissue disorders such as sarcoidosis can include pulmonary fibrosis as part of the disease (Paramothayan, S et al. (2008) Respir Med. 102:1-9), and infectious diseases of the lung can cause fibrosis as a long term consequence of infection, particularly chronic infections. Pulmonary fibrosis is also a side effect of certain medical treatments, particularly radiation therapy to the chest and certain medicines like bleomycin, methotrexate, amiodarone, busulfan, and nitrofurantoin (Catane, R et al. (1979) Int J Radiat Oncol Biol Phys. 5:1513-8; Zisman, D A et al. (2001) Sarcoidosis Vasc Diffuse Lung Dis. 18:243-52; Rakita, L et al. (1983) Am Heart J. 106:906-16; Twohig, K J et al. (1990) Clin Chest Med. 11:31-54; and Witten C M. (1989) Arch Phys Med. Rehabil. 70:55-7).

In addition to the wide array of potential causes of pulmonary fibrosis outlined above, there are a large number of cases of idiopathic pulmonary fibrosis where no clear causal agent or disease can be identified. Increasingly, it appears that genetic factors can play a significant role in these cases of pulmonary fibrosis (Steele, M P et al. (2007) Respiration 74:601-8; Brass, D M et al. (2007) Proc Am Thorac Soc. 4:92-100 and du Bois R M. (2006) Semin Respir Crit. Care Med. 27:581-8).

### 2.5 Bleomycin-Induced Pulmonary Fibrosis in Mice

Bleomycin, a glycopeptides anti-cancer agent originally isolated from Streptomyces verticillus is currently approved for use against squamous cell carcinoma, testicular cancer and Hodgkin's disease. Pulmonary fibrosis is a significant toxicity associated with the use of bleomycin, occurring in a significant percentage of patients undergoing bleomycin therapy. In C57/B6 mice, pulmonary inflammation and subsequent fibrosis is reproducible following bleomycin treatment, and the severity of pulmonary fibrosis is dose-dependent with higher doses being used to evaluate short term inflammatory effects, while lower doses are used to study the longer term impact of pulmonary fibrosis. In this model, mice are given 4 U/kg of bleomycin intranasally and pulmonary fibrosis is evaluated histologically 30 days after the administration of the bleomycin.

This study assessed the effects of the Smoothened inhibitor, IPI-926, on bleomycin-induced pulmonary fibrosis in mice.

### 2.6 Materials and Methods

### Test Article

IPI-926 in 5% wt/wt HPBCD in Water for Injection

### Administration of Test Article

Oral administration by gavage
QOD for 30 days
40 mg/kg
Administered volume varies by weight

### Study Locations

The study was performed at Biomodels AAALAC accredited facility in Watertown, Mass. Approval for this study (approval number 10-0309-1) was obtained from Biomodels IACUC.

### Animals

Thirty-six (36) female C57B1/6 mice (Charles River Laboratories), aged 5 to 6 weeks, with a mean body weight of 19.7 g at study commencement, were used. Animals were individually numbered using an ear punch and housed in groups of eight animals per cage. Animals were acclimatized for 3 days prior to study commencement. During acclimatization, the animals were observed daily in order to reject animals that presented in poor condition.

### Housing

The study was performed in animal rooms provided with filtered air at a temperature of 70° F.+/−5° F., 50+/−20% relative humidity, and set to maintain a minimum of 12 to 15 air changes per hour. The room was on an automatic timer for a light-dark cycle of 12 hours on and 12 hours off with no twilight. Sterile Bed-O-Cobs® bedding was used. Bedding was changed a minimum of once per week. Cages, tops, bottles, etc. were washed with a commercial detergent, rinsed and allowed to air dry. Floors were swept daily and mopped a minimum of twice weekly with a commercial detergent. Walls and cage racks were sponged a minimum of once per month with a dilute bleach solution. The temperature and relative humidity were recorded during the study, and the records retained.

### Diet

Animals were fed with LabDiet 5053 Rodent Diet and water was provided ad libitum.

### Animal Randomization and Allocations

Mice were randomly and prospectively divided into four treatment groups of eight animals each plus a naïve group of four mice. Each animal was identified by an ear punch corresponding to an individual number. A cage card identified each cage or label marked with the study number, treatment group number and animal numbers.

### Fibrosis Induction

Pulmonary fibrosis was induced with a single intra-nasal administration of bleomycin at 4 U/kg in 25 μL of saline. Animals were anesthetized by breathing isoflurane in a closed chamber (2-3% isoflurane 200-300 mls oxygen) to effect. When non-responsive to toe pinch, each animal was held in one hand in an upright position and the bleomycin placed in nares and the droplet watched until inhaled. Animals were then released into a recovery cage.

### BAL Fluid Collection and Evaluation

At necropsy, the inflammatory infiltrated was evaluated by counting cells in the fluid collected following bronchiolar lavage. Bronchiolar lavage was performed by inserting a 20-gauge cannula into the trachea, which was tied in place with suture. The cannula was attached to a 1 mL syringe which was used to deliver and then recover 1 mL of sterile PBS. This fluid was then centrifuged to recover the cells and the resulting fluid saved for potential evaluation. Cell counts were performed on a HTI PCE-90vet automated veterinary hematology analyzer.

### Histology

Each lung sample was trimmed into 1-3 sections. Tissues were embedded in paraffin and sectioned at approximately 5 microns. Two slides for each lung were stained. One was stained with hematoxylin and eosin (H&E) and another serial section was stained with a trichrome stain. All slides were examined by a board-certified veterinary pathologist. Tissues were scored for chronic inflammation and interstitial fibrosis according to the criteria in Table 5 below. In addition the number of foci of interstitial fibrosis in the lung that filled a 10× objective field (100× total magnification) were counted and reported. Statistical analysis was performed with GraphPad Prism version 4.03 and included a Kruskal-Walis test followed by a Dunn's post-test comparing all groups to each other. Individual animal findings are listed herein in the Tables below.

TABLE 5
Histopathologic Scoring Criteria

| | |
|---|---|
| Score | Description |

Chronic Inflammation

| | |
|---|---|
| 0 | None present |
| 1 | Rare foci; minimal |
| 2 | Scattered aggregates or mild diffuse inflammation |
| 3 | Numerous aggregates or moderate diffuse inflammation |
| 4 | More marked inflammation either scattered or diffuse |
| 5 | More severe inflammation either scattered diffuse inflammation |

Interstitial Fibrosis

| | |
|---|---|
| 0 | None present |
| 1 | 1-6 foci that fill a 10x objective field |
| 2 | 7-12 foci that fill a 10x objective field |
| 3 | 13-18 foci that fill a 10x objective field |
| 4 | 19-24 foci that fill a 10x objective field |
| 5 | 25 or more foci that fill a 10x objective field |

### 2.7 Study Design

Thirty-two (32) female C57/B6 mice were randomly and prospectively divided into four groups of eight animals each. Four (4) animals were used as naïve controls for a total of thirty-six (36) mice in the study. Anesthestized mice received a single dose of bleomycin in saline (4 U/kg) intranasally on Day 0. At this dose of bleomyciti, the impact to the anilnals is relatively mild. There is little long term effect on weight, but histological changes ate noticeable after 14 days and the fibrosis progresses with noticeably more fibrosis at days 21 and 28.

Test article or vehicle was given as shown in Table 5. Animals were weighed and evaluated for respiratory distress daily. Animals showing signs of respiratory distress were given diazepam (0.1 ml IP 1 mg/kg twice daily). In one case the respiratory distress could not be alleviated with diazepam and the mouse was euthanized. On Day 30 mice were euthanized by CO₂ asphyxia and the lungs irrigated to evaluate the cells and cytokines present in the BAL fluid, after which one lung from each mouse was frozen for additional analysis and the other fixed in formalin for histology.

TABLE 6
Study Design: INF-01

| | Number | | | |
|---|---|---|---|---|
| | of | | Dose | |
| Group | Animals | Treatment/Route | mg/kg | Dose Schedule |
| 1 | 8♀ | Vehicle | NA | Qod, Day −10 to Day 30 |
| 2 | 8♀ | IPI-926 PO | 40 | Qod, Day −10 to Day 30 |
| 3 | 8♀ | IPI-926 PO | 40 | Qod, Day 0 to Day 30 |
| 4 | 8♀ | IPI-926 PO | 40 | Qod, Day 14 to Day 30 |
| 5 | 4♀ | Naïve | N/A | N/A |

### 2.8 Outcome Evaluation

All animals were weighed and evaluated for respiratory distress daily throughout this study. Respiratory distress was defined as an increase in respiratory rate and/or obvious respiratory effort. Animals showing signs of respiratory distress were given diazepam (0.1 ml IP 1 mg/kg bid) and euthanized if the respiratory distress was not alleviated with diazepam.

### Cellularity of BAL Fluids and Cytokine Level

At sacrifice, BAL fluids were collected from all animals. The cells from these fluids were evaluated for differential white count. The fluids were retained for cytokine analysis.

### Histology

Lungs were taken at sacrifice on Day 30 for analysis. Lungs were removed and partitioned in two halves. On half was fixed in 10% formalin, before embedding and sectioning. Slides made from each sample were stained with H&E or Masson's Trichrome. The other half of lung sample were snap-frozen in liquid nitrogen and stored at −80 C for future analysis of mRNA by QT-PCR and/or proteomic analysis.

### Animal Weights

All animals were weighed daily throughout this study. Group weight change was expressed as a daily group mean weight. Animals that lose greater than 20% of their total starting body weight on any given day were euthanized. The daily measurement of animal weight is summarized in Table 7 below.

### 2.9 Results and Discussion

### Survival

Two animals died or were euthanized during this study. Animal 13 in the group treated with IPI926 from Day −10 died on Day 2, and animal 16 in the same group was euthanized on Day 23 for poor condition. Samples were collected from animal 16, but not from animal 13.

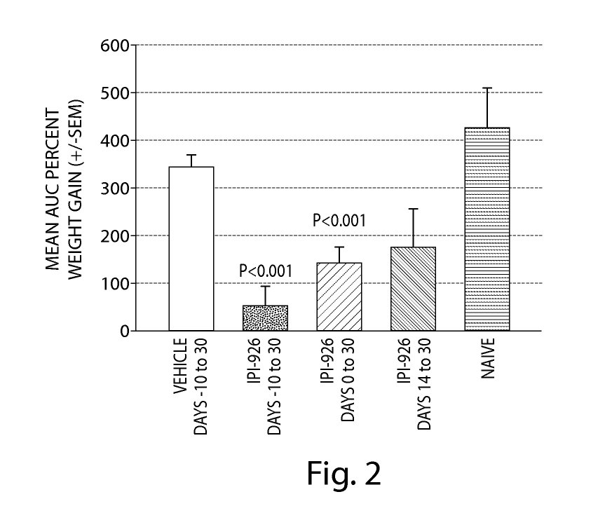

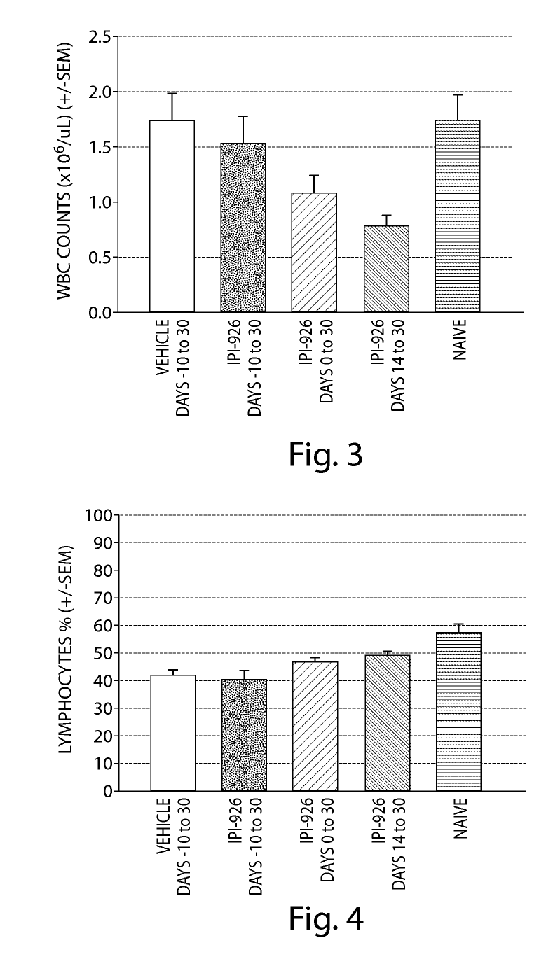

### Weight Change (FIGS. 2 and 3)

The mean daily percent weight gains for the all treatment groups are shown in FIG. 2. Naïve mice gained an average of 19.4% of their starting weight during the study. Mice that were treated with bleomycin plus vehicle gained an average of 16.1% of their starting weights through the course of the study. Mice treated with bleomycin plus IPI-926 on Days 0 to 30 gained an average of 6.7% of their starting weight during the study. Mice treated with bleomycin plus IPI-926 on Days −10 to 30 gained an average of 9.8% of their starting weight during the study. Mice treated with bleomycin plus IPI-926 on Days 14 to 30 gained an average of 11.6% of their starting weight during the study.

The significance of these differences was evaluated by calculating the area-under-the-curve (AUC) for the weight gain of each animal, and then comparing the different treatment groups using a One-Way ANOVA test. The results of this analysis indicated that there were significant differences between the vehicle control group and the groups treated with IPI-926 on Days −10 to 30 (p<0.001), and Days 0 to 30 (p<0.001).

### Bronchiolar Lavage Counts (FIGS. 4-7)

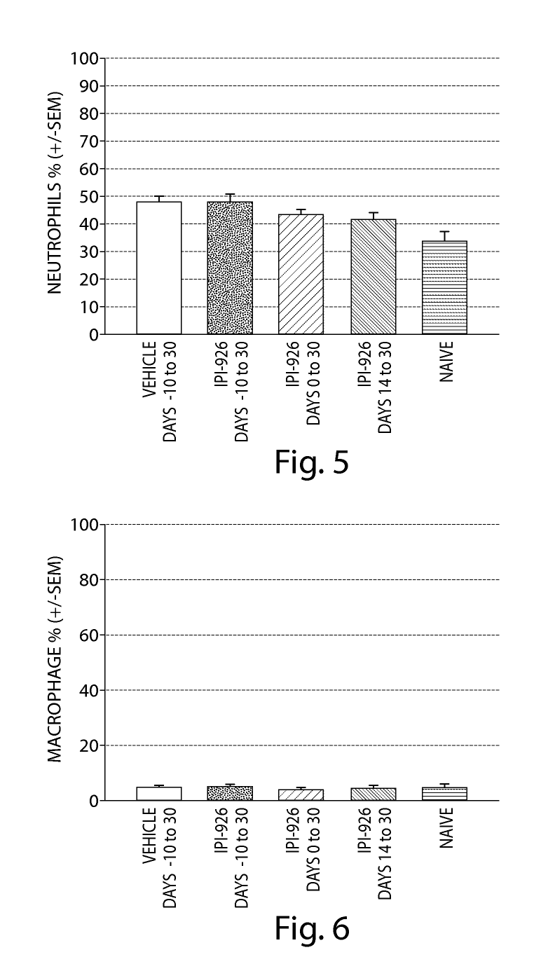

At sacrifice, lungs from all animals were lavaged with saline, and the cells present in the resulting bronchiolar lavage fluid were quantified using a HTI PCE-90vet automated veterinary hematology analyzer. Total white blood cell (WBC) counts are shown in FIG. 4. The naïve animals had mean WBC counts of 1.74×10⁶ cells/μL, compared to WBC counts of 1.75×10⁶ cells/uL in the Group treated with bleomycin and vehicle. The group treated with IPI 926 from Day −10 to Day 30 had a mean WBC counts of 1.53×10⁶ cells/μL, while the mice in the groups treated with IPI926 on Days 0-30 and 14-20 had mean WBC counts of 1.09×10⁶ cells/μL and 0.79×10⁶ cells/μL respectively. The percentages of neutrophils, lymphocytes and macrophage are shown FIGS. 5, 6 and 7, respectively). Naïve mice had mean counts of 34% neutrophils, 58% lymphocytes and 1.8% monocytes. Mice treated with bleomycin and vehicle had mean counts of 48% neutrophils, 42% lymphocytes and 1.7% monocytes. Mice treated with bleomycin and IPI-926 from Day −10 to 30 had counts that were virtually identical to the vehicle controls (48% neutrophils, 41% lymphocytes and 1.5% monocytes). The groups treated with IPI-926 from Day 0 to 30 or Day 14 to 30 had counts that were closer to the naïve animals (42-44% neutrophils, 47-49% lymphocytes and 08-1.1% monocytes). Table 9, below, summarizes the total white blood cell (WBC) count in bronchiolar lavage fluid samples for each of the treatment animal groups according to Table 6.

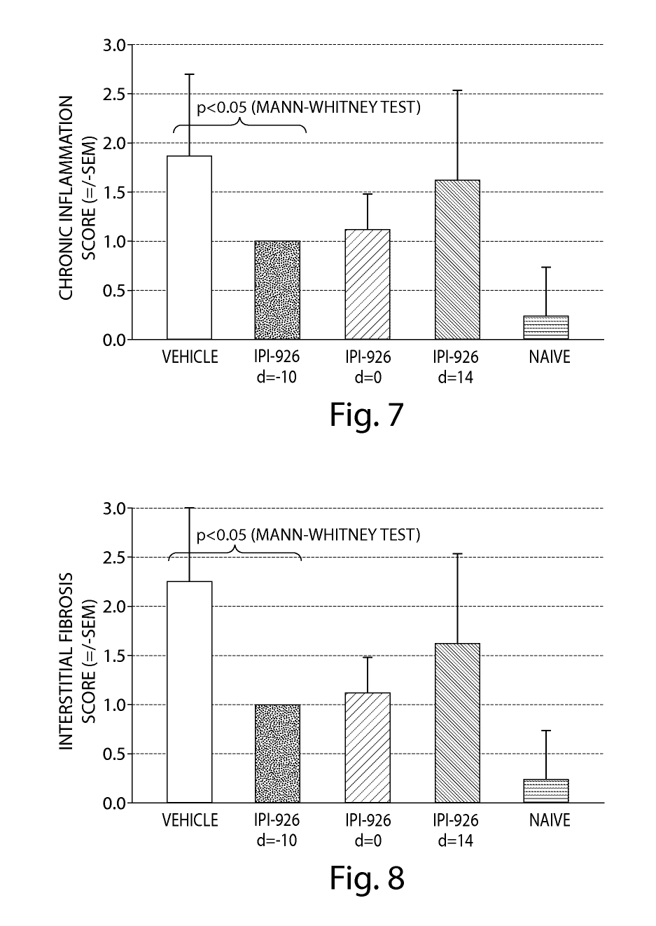

### Histology (FIGS. 7-10)

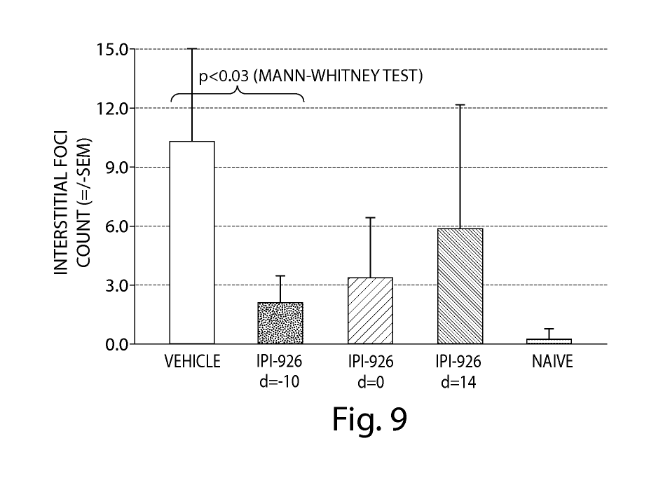

Slides from each lung were stained with H&E and Masson's Trichrome. The H&E stained slides were used for an initial overall evaluation and analysis of inflammation, while the Masson's Trichrome stained slides were used for a detailed analysis of the fibrosis in each lung. Two parameters, chronic inflammation and interstitial fibrosis were scored according to the scoring scales shown in Table 5. The mean data for chronic inflammation is shown in FIG. 8 and for interstitial fibrosis in FIG. 9. Statistical analysis was performed according to the Mann-Whitney test. These results demonstrate that at d=−10, IPI-926 treatment of animals actually decreases the amount of lung fibrosis induced by bleomycin in a statistically significant manner (p<0.05). Prophylactic intervention at d=−10 prior to bleomycin treatment resulted in significant decreases in fibrotic disease.

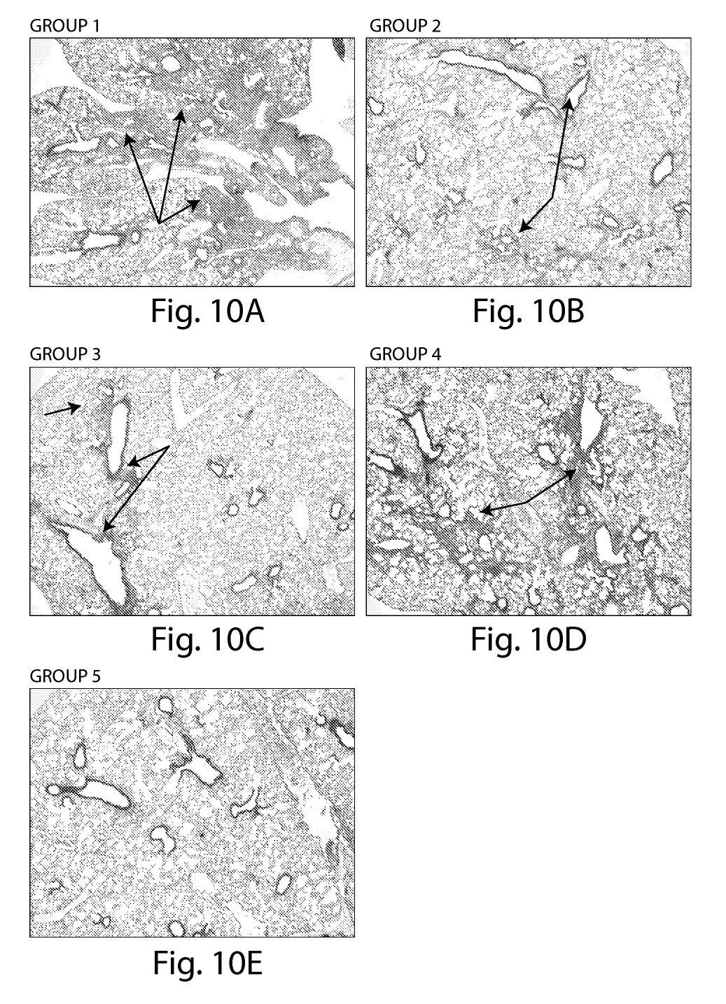

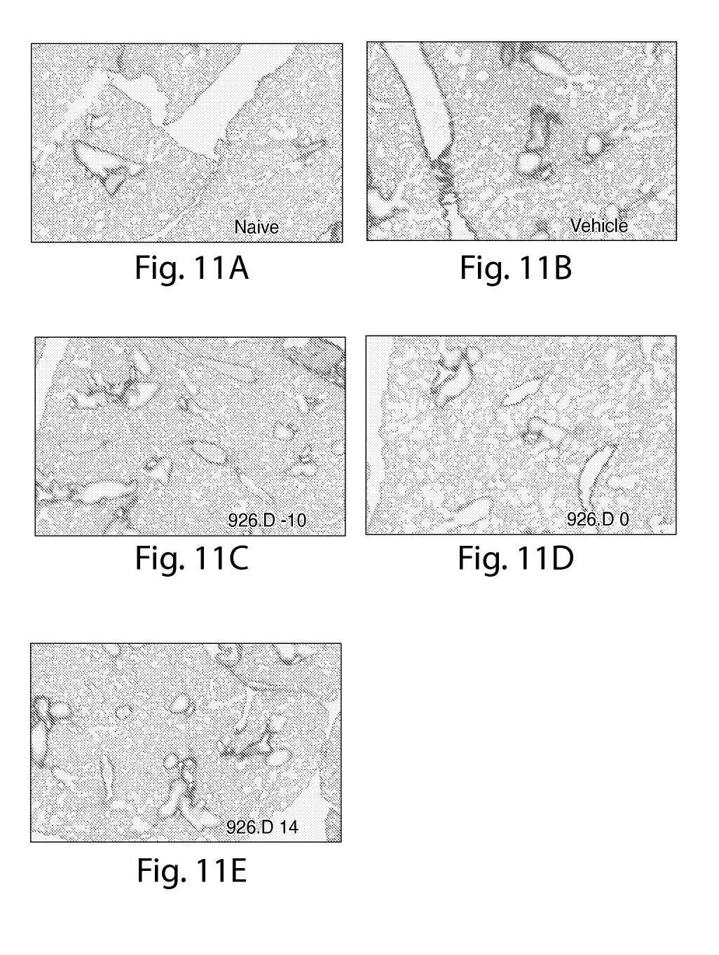

In addition, the numbers of foci of interstitial fibrosis were enumerated and the results of this analysis are shown in FIGS. 10A-10E. Representative photographs of sections of lung are shown in FIGS. 11A-11E. These are characterized by chronic peribronchiolar and interstitial inflammation composed of lymphocytes, plasma cells and macrophages. These inflamed regions are accompanied by interstitial fibrosis. These changes vary in severity across samples. Naive mice lungs were essentially normal.

### Chronic Inflammation

Mice in the group treated with bleomycin plus vehicle had a mean chronic inflammation score of 1.9 compared to 0.25 in the naïve mice. Mice treated with IPI926 from Day −10 to Day 30 had mean chronic inflammation scores of 1.0, compared with mean scores of 1.1 and 1.6 for the groups treated with IPI926 from Days 0 to 30 and 14 to 30 respectively. The differences between the vehicle control and naïve mice were statistically significant in Kruskal-Wallis One Way Analysis of Variance on Ranks test (p=0.003). The groups treated with IPI926 did not show a statistically significant reduction in chronic inflammation relative to the vehicle controls, however the differences between the IPI926 treated groups and the naïve mice were not statistically significant either.

### Interstitial Fibrosis

Mice in the group treated with bleomycin plus vehicle had a mean interstitial fibrosis score of 2.25 compared to 0.25 in the naïve mice. Mice treated with IPI926 from Day −10 to Day 30 had mean interstitial fibrosis scores of 1.0, compared with mean scores of 1.1 and 1.6 for the groups treated with IPI926 from Days 0 to 30 and 14 to 30 respectively. The differences between the vehicle control and naïve mice were statistically significant in Kruskal-Wallis One Way Analysis of Variance (ANOVA) on Ranks test (p=0.003). The groups treated with IPI926 did not show a statistically significant reduction in interstitial fibrosis relative to the vehicle controls, however the differences between the IPI926 treated groups and the naïve mice were not statistically significant either.

### Number of Foci of Interstitial Fibrosis

Mice in the group treated with bleomycin plus vehicle had a mean number of interstitial fibrosis foci of 10.4 compared to 0.25 in the naïve mice. Mice treated with IPI926 from Day −10 to Day 30 had mean interstitial fibrosis foci counts of 2.1, compared with foci totals of 3.4 and 5.9 for the groups treated with IPI926 from Days 0 to 30 and Days 14 to 30 respectively. The differences between the vehicle control and naïve mice were statistically significant in Kruskal-Wallis One Way Analysis of Variance (ANOVA) on Ranks test (p=0.006). The groups treated with IPI926 did not show a statistically significant reduction in the number of foci of interstitial fibrosis relative to the vehicle controls, however the differences between the IPI926 treated groups and the naïve mice were not statistically significant either. The number of foci for interstitial fibrosis for each animal is summarized in Table 8 below.

FIGS. 11A-11E are representative photomicrographs of lung samples from Example 2 as stained using PicroSiruis Red to stain connective tissues. Panels A-E correspond to Naïve, Vehicle, and treated samples for 10, 0 and 14 days, respectively. Picosirius Red stains connective tissue, and is thus used to measure fibrotic infiltration.

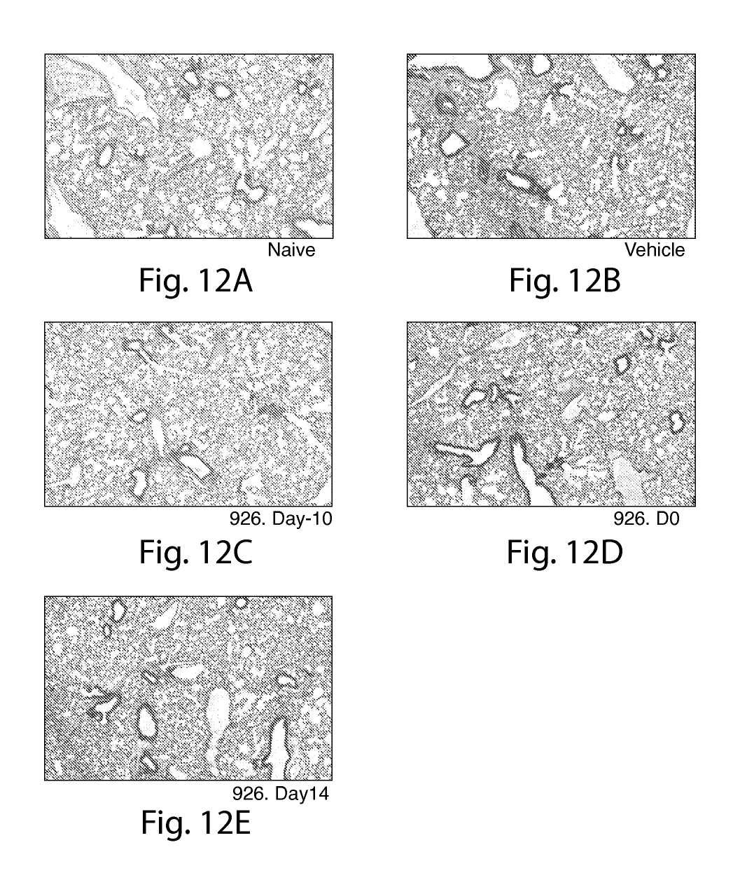

FIGS. 12A-12E are representative photomicrographs of lung samples from Example 2 as stained using a murine sonic hedgehog (SHH) antibody. Panels A-E correspond to Naïve, Vehicle, and treated samples for 10, 0 and 14 days, respectively.

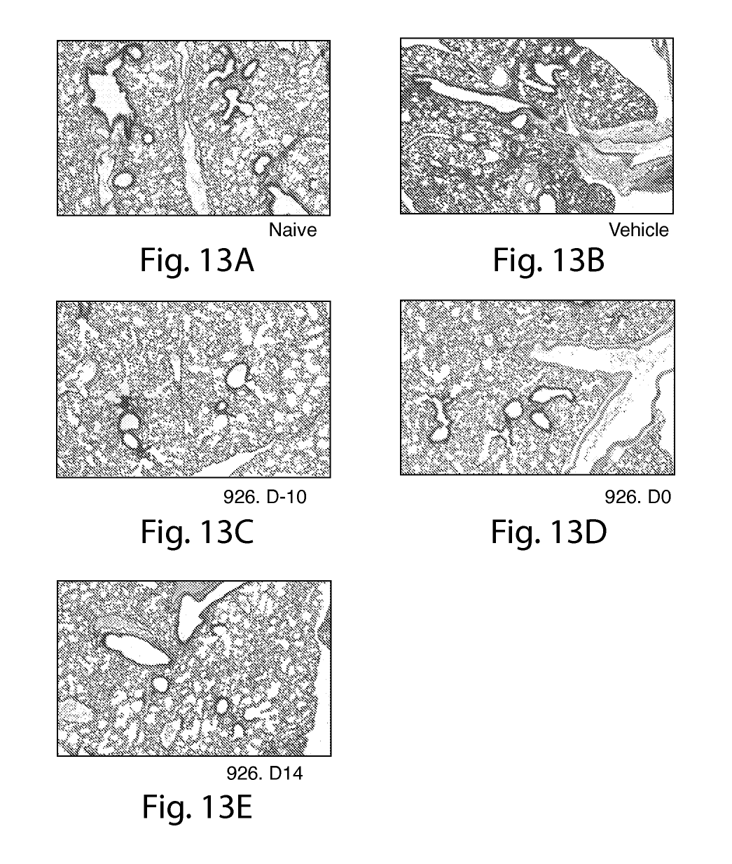

FIGS. 13A-13E are representative photomicrographs of lung samples from Example 3 as stained using a murine GLi1 antibody. Panels A-E correspond to Naïve, Vehicle, and treated samples for 10, 0 and 14 days, respectively.

FIGS. 12 and 13 show immunohistochemistry of tissue samples from the Bleomycin lung fibrosis study stained for Sonic Hedgehog (SHh) and Gli-1. The results indicate that in response to bleomycin (vehicle treated), both SHh and Gli-1 are upregulated, which correlates with induction of fibrosis (increased picosirius red). Treatment with IPI-926 appears to decrease the Gli-1 immunoreactivity at d=−10 (IPI-926 given 10 days prior to the start of bleomycin administration), d=0 (IPI-926 given in conjunction with start of bleomycin administration) and d=14 (IPI-926 given 14 days after the start of bleomycin administration) in comparison to vehicle treatment. In regards to SHh immunoreactivity, only IPI-926 administered at d=−10 appeared to have an effect on this signal. Also, only the decrease in SHh and Gli-1 at day=−10 correlated with a decrease in picosirius red reactivity (fibrosis).

### Summary

1. Significant reductions in weight gain were seen in animals treated with IPI926 on alternating days from Day −10 to Day 30, or from Day 0 to Day 30 relative to vehicle controls. In addition, 2 mice in the group treated with IPI926 from Day −10 to Day 30 died on Days 13 and 16.

2. Evaluation of the bronchiolar lavage fluids for cellularity revealed no substantial differences between the naïve animals and the control group treated with bleomycin and vehicle. Reductions in white cell count were observed in all groups treated with IPI926, with the greatest reductions being seen in the group treated from Day 14 to Day 30.

3. Under the conditions of the study, treatment of a bleomycin-induced pulmonary fibrosis model in mice with IPI-926 at 40 mg/kg results in a reduction in pulmonary fibrosis and associated inflammation compared to the vehicle control group. The dosing regimen from Day-10 to Day 30 shows the greatest effect in reducing pulmonary fibrosis in the model.

TABLE 7
Daily Animal Weights

| | | Day | | | | | | | | | | |
|---|---|---|---|---|---|---|---|---|---|---|---|---|
| Group | Animal | 11 | 12 | 13 | 14 | 15 | 16 | 17 | 18 | 19 | 20 | 21 |
| 1 | 1 | 19.71 | 20.11 | 20.89 | 20.4 | 20.1 | 20.87 | 20.27 | 20.16 | 20.58 | 20.86 | 20.75 |
| 1 | 2 | 20.55 | 21.39 | 21.68 | 22.28 | 21.54 | 21.71 | 21.37 | 21.02 | 21.43 | 21.31 | 21.54 |
| 1 | 3 | 21.91 | 21.78 | 21.77 | 22.3 | 22.3 | 22.32 | 21.63 | 21.53 | 21.93 | 22.43 | 22.73 |
| 1 | 4 | 21.8 | 20.86 | 20.71 | 21.49 | 21.58 | 22.69 | 21.32 | 21.33 | 21.76 | 22.84 | 22.27 |
| 1 | 5 | 20.37 | 20.37 | 20.21 | 20.98 | 21.01 | 20.84 | 20.97 | 20.55 | 20.98 | 21.34 | 21.14 |
| 1 | 6 | 20.88 | 20.25 | 20.39 | 20.88 | 20.92 | 20.62 | 20.44 | 20.24 | 20.67 | 21.75 | 21.2 |
| 1 | 7 | 22.04 | 21.47 | 21.59 | 22.4 | 21.97 | 23.02 | 22.2 | 21.74 | 22.13 | 23.18 | 23.18 |
| 1 | 8 | 19.4 | 19.54 | 19.55 | 19.56 | 19.64 | 20.32 | 20.47 | 19.74 | 20.18 | 20.42 | 20.17 |
| 2 | 9 | 19.6 | 20.12 | 19.96 | 20.01 | 19.49 | 20.63 | 20.19 | 20.06 | 20.44 | 20.08 | 20.33 |
| 2 | 10 | 20.54 | 21.32 | 21.03 | 20.94 | 20.5 | 20.72 | 20.99 | 20.92 | 21.31 | 21.11 | 20.79 |
| 2 | 11 | 20.08 | 19.71 | 20.15 | 20 | 20.47 | 20.3 | 20.66 | 19.96 | 20.37 | 20.01 | 20.5 |
| 2 | 12 | 20.31 | 19.92 | 20.22 | 20.49 | 20.82 | 20.9 | 20.71 | 20.02 | 20.41 | 20.64 | 21.26 |
| 2 | 13 | | | | | | | | | | | |
| 2 | 14 | 20.67 | 21.01 | 20.79 | 20.36 | 20.17 | 21 | 21.06 | 20.54 | 20.93 | 21.08 | 19.19 |
| 2 | 15 | 16.85 | 17.94 | 18.72 | 19.59 | 19.87 | 20.25 | 19.42 | 19.71 | 20.14 | 20.27 | 20.63 |
| 2 | 16 | 20.33 | 20.98 | 19.64 | 19.83 | 20.08 | 20.89 | 20.45 | 20.24 | 20.61 | 20.26 | 17.76 |
| 3 | 17 | 20.38 | 20.67 | 20.48 | 21.06 | 20.91 | 20.93 | 21.11 | 20.46 | 20.78 | 21.17 | 21.23 |
| 3 | 18 | 19.82 | 19.77 | 19.96 | 21.19 | 20.65 | 21.06 | 20.6 | 20 | 20.39 | 21.22 | 20.89 |
| 3 | 19 | 19.74 | 19.48 | 19.73 | 20.54 | 20.48 | 20.55 | 20.13 | 19.9 | 20.19 | 21 | 20.95 |
| 3 | 20 | 20.74 | 20.66 | 20.48 | 20.01 | 20.51 | 20.88 | 21.24 | 20.8 | 21.18 | 20.78 | 20.82 |
| 3 | 21 | 20.21 | 19.24 | 19.38 | 19.47 | 20.33 | 20.3 | 20.42 | 20.07 | 20.33 | 20.13 | 20.43 |
| 3 | 22 | 20.71 | 21.31 | 21.18 | 20.86 | 20.43 | 20.84 | 21.26 | 21.35 | 21.71 | 21.58 | 21.4 |
| 3 | 23 | 20.68 | 20.03 | 19.74 | 19.89 | 20 | 19.9 | 20.33 | 20.06 | 20.46 | 20.37 | 20.3 |
| 3 | 24 | 20.37 | 20.31 | 20.52 | 21.09 | 20.64 | 20.75 | 21.12 | 20.92 | 21.34 | 21.51 | 21 |
| 4 | 25 | 22.14 | 22.57 | 21.81 | 23.4 | 22.72 | 23.24 | 23.06 | 21.93 | 22.31 | 23.56 | 23.68 |
| 4 | 26 | 21.07 | 20.68 | 20.02 | 20.73 | 20.51 | 20.79 | 21.3 | 20.52 | 20.93 | 21.14 | 21.55 |
| 4 | 27 | 16.37 | 16.71 | 15.95 | 16.03 | 15.84 | 16.79 | 17.21 | 17.35 | 17.78 | 17.31 | 17.95 |
| 4 | 28 | 21.11 | 21.32 | 21.72 | 21.8 | 21.4 | 21.83 | 21.4 | 21.23 | 21.61 | 21.61 | 21.68 |
| 4 | 29 | 19.56 | 19.44 | 19.09 | 20.26 | 20.48 | 20.98 | 20.5 | 19.85 | 20.24 | 20.58 | 20.78 |
| 4 | 30 | 20.47 | 20.16 | 20.01 | 20.52 | 19.98 | 21.22 | 20.58 | 20.13 | 20.47 | 21.02 | 20.7 |
| 4 | 31 | 21.62 | 21.43 | 21.89 | 22.58 | 21.7 | 22 | 21.84 | 21.83 | 22.13 | 21.94 | 22.09 |
| 4 | 32 | 21.82 | 22.05 | 22.03 | 22.79 | 22.64 | 22.88 | 22.29 | 21.7 | 22.19 | 22.13 | 22.44 |
| 5 | 33 | 21.68 | 21.61 | 23.09 | 22.7 | 22.61 | 22.8 | 22.64 | 22.08 | 22.44 | 22.06 | 21.96 |
| 5 | 34 | 22.69 | 22.36 | 22.53 | 22.92 | 23.01 | 22.82 | 22.67 | 22.96 | 23.33 | 23.43 | 23.46 |
| 5 | 35 | 20.62 | 21.13 | 21.16 | 21.17 | 21.59 | 21.83 | 21.97 | 21.44 | 21.79 | 21.35 | 21.4 |
| 5 | 36 | 21.82 | 22.05 | 22.75 | 23.09 | 23.07 | 22.43 | 22.26 | 23.34 | 23.71 | 22.95 | 23.19 |

| | | Day | | | | | | | | |
|---|---|---|---|---|---|---|---|---|---|---|
| Group | Animal | 22 | 23 | 24 | 25 | 26 | 27 | 28 | 29 | 30 |
| 1 | 1 | 20.99 | 21.07 | 20.04 | 20.36 | 20.51 | 21.09 | 21.33 | 20.99 | 20.54 |
| 1 | 2 | 21.44 | 22.01 | 21.51 | 21.47 | 21.23 | 21.69 | 22.19 | 21.7 | 22.2 |
| 1 | 3 | 22.88 | 23.21 | 22.28 | 22.28 | 22.35 | 22.69 | 23.19 | 22.83 | 22.88 |
| 1 | 4 | 22.38 | 22.54 | 22.51 | 22.69 | 22.74 | 22.1 | 22.59 | 22.52 | 23 |
| 1 | 5 | 21 | 21.18 | 21.07 | 21.39 | 21.41 | 21.33 | 21.27 | 21.6 | 21.63 |
| 1 | 6 | 21.2 | 21.39 | 21.6 | 22.08 | 21.28 | 22.07 | 22.72 | 22.39 | 22.62 |
| 1 | 7 | 22.17 | 22.48 | 22.2 | 22.46 | 22.69 | 22.35 | 21.45 | 22.69 | 23.32 |
| 1 | 8 | 20.73 | 21.01 | 20.3 | 20.41 | 20.59 | 20.73 | 21.06 | 20.77 | 20.74 |
| 2 | 9 | 20.48 | 20.44 | 20.48 | 20.43 | 20.74 | 20.8 | 20.7 | 20.98 | 20.96 |
| 2 | 10 | 21.5 | 22.11 | 22.23 | 21.7 | 21.59 | 21.51 | 21.92 | 21.47 | 22.15 |
| 2 | 11 | 20.84 | 20.56 | 20.44 | 20.53 | 20.78 | 20.61 | 20.58 | 20.75 | 21.47 |
| 2 | 12 | 21.23 | 20.88 | 21.08 | 21.19 | 21.22 | 21.03 | 21.07 | 21 | 21.61 |
| 2 | 13 | | | | | | | | | |
| 2 | 14 | 19.94 | 20.45 | 20.74 | 20.76 | 20.42 | 20.55 | 20.82 | 20.46 | 21.25 |
| 2 | 15 | 21.46 | 20.04 | 20.56 | 20.47 | 20.61 | 20.76 | 21.16 | 21.08 | 21.17 |
| 2 | 16 | 17.5 | | sac'd | | | | | | |
| 3 | 17 | 21.07 | 21.03 | 20.92 | 20.84 | 21.04 | 21.28 | 21.16 | 20.46 | 21.54 |
| 3 | 18 | 21.15 | 21.36 | 20.71 | 20.6 | 21.15 | 21.61 | 21.2 | 21.61 | 22.13 |
| 3 | 19 | 20.75 | 20.44 | 20.64 | 20.94 | 20.73 | 21.12 | 20.95 | 21.37 | 21.8 |
| 3 | 20 | 21.04 | 21.04 | 21.12 | 20.97 | 20.98 | 21.07 | 21.27 | 21.03 | 21.68 |
| 3 | 21 | 20.48 | 20.63 | 19.82 | 20.31 | 20.46 | 20.65 | 20.69 | 20.58 | 21.03 |
| 3 | 22 | 21.74 | 22.04 | 22.03 | 21.73 | 21.87 | 22.22 | 21.58 | 21.94 | 22.56 |
| 3 | 23 | 20.27 | 20.36 | 20.45 | 20.33 | 20.21 | 20.49 | 20.32 | 20.5 | 20.7 |
| 3 | 24 | 21.14 | 21.36 | 21.52 | 21.38 | 21.21 | 21.09 | 21.12 | 21.3 | 21.68 |
| 4 | 25 | 23.04 | 23.47 | 23.93 | 23.49 | 24.21 | 23.39 | 23.02 | 23.06 | 23.75 |
| 4 | 26 | 20.65 | 21.54 | 21.74 | 21.2 | 21.22 | 20.7 | 21.22 | 21.29 | 21.75 |
| 4 | 27 | 18.35 | 19.53 | 19.36 | 19.32 | 18.97 | 19.66 | 19.57 | 19.85 | 19.93 |
| 4 | 28 | 21.71 | 22.04 | 21.57 | 21.37 | 21.12 | 21.77 | 21.69 | 21.74 | 22.27 |
| 4 | 29 | 20.92 | 20.18 | 20.54 | 20.58 | 20.81 | 20.99 | 20.82 | 20.98 | 21.69 |
| 4 | 30 | 20.29 | 20.64 | 21.12 | 20.55 | 21.07 | 21.43 | 21.47 | 21.06 | 21.46 |
| 4 | 31 | 22.51 | 22.37 | 22.66 | 22.21 | 22.36 | 21.94 | 21.67 | 21.97 | 22.39 |
| 4 | 32 | 22.25 | 22.16 | 22.63 | 22.26 | 22.59 | 22.8 | 22.26 | 22.27 | 22.7 |
| 5 | 33 | 22.42 | 22.32 | 22.06 | 22.55 | 23.19 | 23.27 | 23.41 | 23.01 | 23.2 |
| 5 | 34 | 23.3 | 24.1 | 24.1 | 24.1 | 24.38 | 24.57 | 24.96 | 24.72 | 24.29 |
| 5 | 35 | 21.28 | 21.78 | 21.5 | 21.5 | 21.96 | 21.7 | 21.89 | 22.19 | 22.47 |
| 5 | 36 | 23.02 | 23.67 | 23.15 | 23.15 | 24.13 | 24.01 | 24.07 | 23.93 | 23.73 |

TABLE 8
Histopathology Data

| | Chronic | Number of Foci of | | |
|---|---|---|---|---|
| | Inflammation | Interstitial Fibrosis that Fill | Interstitial | |
| Animal # | (0-5) | a 10x Objective Field | Fibrosis (0-5) | Comment |
| 1 | 2 | 7 | 2 | |
| 2 | 1 | 1 | 1 | |
| 3 | 3 | 32 | 5 | Many macrophages with |
| | | | | cholesterol crystals |
| 4 | 2 | 7 | 2 | |
| 5 | 2 | 4 | 2 | |
| 6 | 1 | 5 | 1 | |
| 7 | 3 | 24 | 4 | |
| 8 | 1 | 3 | 1 | |
| 9 | 1 | 2 | 1 | |
| 10 | 1 | 1 | 1 | |
| 11 | 1 | 2 | 1 | |
| 12 | 1 | 1 | 1 | |
| 14 | 1 | 1 | 1 | |
| 15 | 1 | 4 | 1 | |
| 16 | 1 | 4 | 1 | |
| 17 | 1 | 2 | 1 | |
| 18 | 1 | 2 | 1 | |
| 19 | 2 | 10 | 2 | |
| 20 | 1 | 1 | 1 | |
| 21 | 1 | 2 | 1 | |
| 22 | 1 | 4 | 1 | |
| 23 | 1 | 5 | 1 | |
| 24 | 1 | 1 | 1 | |
| 25 | 1 | 2 | 1 | |
| 26 | 3 | 14 | 3 | |
| 27 | 1 | 2 | 1 | |
| 28 | 1 | 2 | 1 | |
| 29 | 3 | 17 | 3 | |
| 30 | 2 | 7 | 2 | |
| 31 | 1 | 1 | 1 | |
| 32 | 1 | 2 | 1 | |
| 33 | 1 | 0 | 0 | |
| 34 | 0 | 1 | 1 | |
| 35 | 0 | 0 | 0 | |
| 36 | 0 | 0 | 0 | |

TABLE 9
Bronchiolar lavage fluid counts

| Group | Animal | WBC | % lymph | % neutro | % monos |
|---|---|---|---|---|---|
| 1 | 1 | 0.8 | 47 | 44 | 2 |
| 1 | 2 | 0.9 | 42 | 47 | 5 |
| 1 | 3 | 1.2 | 42 | 42 | 10 |
| 1 | 4 | 2.5 | 45 | 49 | 3 |
| 1 | 5 | 2.1 | 45 | 48 | 4 |
| 1 | 6 | 2.5 | 38 | 56 | 1 |
| 1 | 7 | 2.3 | 31 | 56 | 8 |
| 1 | 8 | 1.6 | 48 | 42 | 5 |
| 2 | 9 | 0.6 | 43 | 43 | 4 |
| 2 | 10 | 1.3 | 44 | 41 | 6 |
| 2 | 11 | 2.1 | 41 | 48 | 8 |
| 2 | 12 | 1.9 | 26 | 60 | 3 |
| 2 | 13 | | | | |
| 2 | 14 | 2.1 | 46 | 49 | 4 |
| 2 | 15 | 1.2 | 44 | 47 | 6 |
| 2 | 16 | | | | |
| 3 | 17 | 1.3 | 41 | 43 | 4 |
| 3 | 18 | 0.1 | 45 | 47 | 3 |
| 3 | 19 | 0.9 | 46 | 44 | 7 |
| 3 | 20 | 1.5 | 40 | 52 | 3 |
| 3 | 21 | 1.1 | 46 | 41 | 4 |
| 3 | 22 | 1.3 | 56 | 34 | 4 |
| 3 | 23 | 1.3 | 51 | 42 | 5 |
| 3 | 24 | 1.2 | 49 | 45 | 4 |
| 4 | 25 | 1.1 | 51 | 42 | 3 |
| 4 | 26 | 0.9 | 53 | 28 | 9 |
| 4 | 27 | 0.4 | 54 | 41 | 3 |
| 4 | 28 | 0.4 | 42 | 50 | 4 |
| 4 | 29 | 0.9 | 55 | 35 | 1 |
| 4 | 30 | 1.1 | 46 | 49 | 2 |
| 4 | 31 | 0.9 | 45 | 43 | 11 |
| 4 | 32 | 0.6 | 49 | 45 | 3 |
| 5 | 33 | 1.9 | 61 | 34 | 5 |
| 5 | 34 | 1.1 | 63 | 31 | 2 |
| 5 | 35 | 1.9 | 49 | 43 | 7 |
| 5 | 36 | 2.1 | 57 | 28 | 6 |

### Example 2B

### Efficacy of IPI 926 on the Severity of Pulmonary Fibrosis Induced by Bleomycin in C57BL/6 Mice

This Example further evaluates the efficacy of IPI 926 on the severity of pulmonary fibrosis induced by bleomycin, and demonstrates a protective effect of IPI-926 administration in this model.

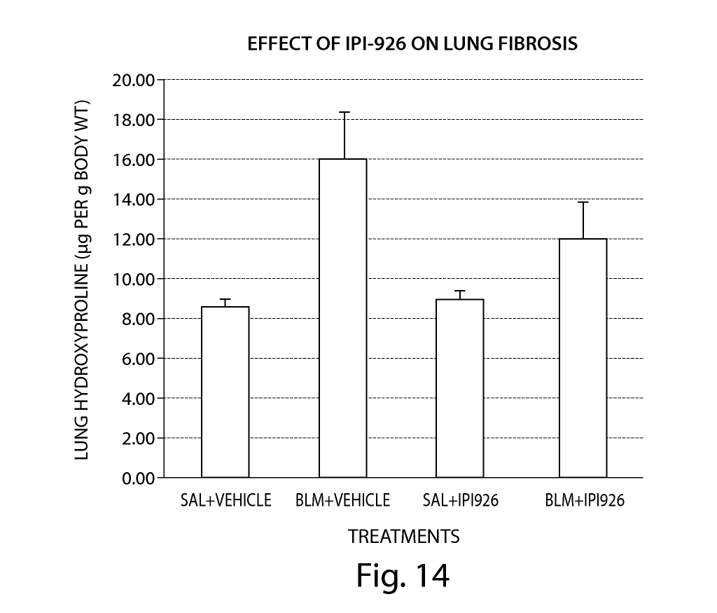

Experimental conditions used in this study are similar to the ones described in Example 2A. FIG. 14 shows the effect of IPI-926 treatment begun on the day of bleomycin insult, as assessed by ug of hydroxproline (a constituent of fibrotic plaques and an accurate measure of fibrosis) per g of animal body weight. Mice were treated with bleomycin (BLM) on day 0 and controls received saline (SAL). Treatment with IPI-926 reduces the fibrosis caused by bleomycin.

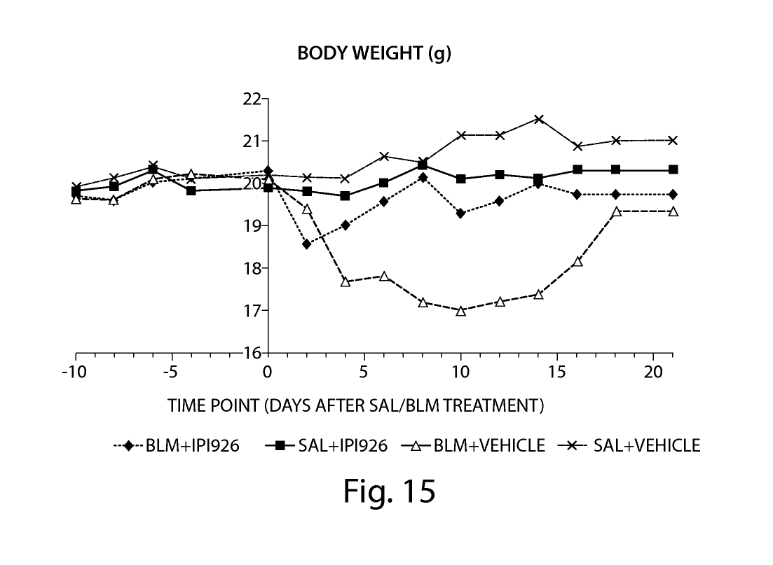

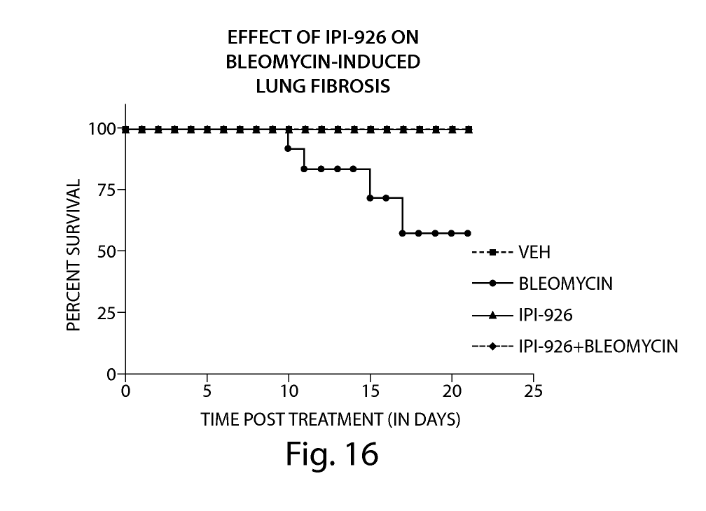

The effects of IPI-926 in body weight and survival that accompany bleomycin insult are shown in FIGS. 15 and 16, respectively. IPI-926 reduces the dramatic loss in body weight that accompanies bleomycin insult. IPI-926 significantly abrogates the increase in animal death that also results from bleomycin administration. Taken together, these results show decreased fibrosis in response to IPI-926. In addition, these results show a protective effect of IPI-926 administration in this lung fibrosis model, as evidenced by decreased animal weight loss and increased survival.

### Example 3

### Liver Fibrosis Induced by Carbon Tetrachloride (CCL4) in BALB/C Mice

This Example describes experimental conditions to test the effects of IPI-926 in murine models of hepatic fibrosis.

### 3.1 Background

There is increasing evidence that the Hedgehog pathway is involved in liver fibrosis. Hh ligands have been shown to be released from injured hepatocytes, and shown to act on the hepatic stellate, cell/myofibroblast compartment to induce transdifferentiation, activation and increased production of ECM.

Applicants have produced a large body of evidence demonstrating that modification of supporting cells or “stroma” by a hedgehog inhibitor, e.g., IPI-926, can affect disease outcome. Indeed, in the KPC mouse model of pancreatic adenocarcinoma, IPI-926 treatment resulted in inhibition of desmoplastic infiltration, decreased metastasis and increased survival.

Certain reports have shown that biliary obstruction in bile duct-ligated rodents (BDL) (as in humans with cholangiopathies) triggers proliferation of bile ductular cells that are surrounded by fibrosis produced by adjacent myofibroblastic cells in the hepatic mesenchyme (Omenetti, A. et al. (2007) Laboratory Investigation 87:499-514). Biliary duct obstruction in BDL mice induces activity of the hedgehog pathway as described by Omenetti, A. et al. (2007) supra. These results provide evidence that the Hh pathway plays a role in adult liver repair, including repair associated with bile duct injuries and cholangiopathies.

Moreover, prophylactic IPI-926 administration in a mouse model of experimental liver metastasis results in dramatic effects on metastatic spread and survival, suggesting that “priming” of the “pre-metastatic niche” in the normal liver can have profound effects on disease spread.

Taken together, these data support the conclusion that IPI-926 intervention can potentially affect disease, such as pancreatic cancer and metastatic spread, where stromal activity and infiltration can translate to other disease states, such as liver fibrosis, where stromal activation is important for disease progression.

### 3.2 Models for Liver Fibrosis

Experimental models for liver fibrosis are available in the art. Applicants describe herein two different animal models that can be used to evaluate effect of IPI-926 in hepatic fibrosis.
(i) Carbontetrachloride-induced hepatic fibrosis: Mice are treated with a CCl4 (0.5 ul/g, dissolved in corn oil) every three days for a total of 4 injections. Mice will be sacrificed 48 h after the last injection of CCl4. During the entire course, mice will be treated with IPI-926 at 40 mg/kg, QOD by oral administration. Each treatment group (IPI-926 and vehicle) will consist of 10 mice. In addition, control groups of 5 mice will receive corn oil and either IPI-926 or vehicle.
(ii) Bile duct ligation: The common bile duct is ligated and mice will be sacrificed after 15 days. During the entire course, mice will be treated with IPI-926 at 40 mg/kg, QOD by oral administration. Each treatment group (IPI-926 and vehicle) will consist of 10 mice. In addition, control groups of 5 mice will undergo sham operation and receive either IPI-926 or vehicle. For additional description of this model, please see Omenetti, A. et al. (2007) supra.
   The treatment scheduled can be altered to assess the effects of prophylactic IPI-926 administration on fibrotic induction. In these studies, IPI-926 can be administered 7 to 14 days prior to the induction of disease by CCl4 treatment or bile duct ligation respectively.
   IPI-926 activity can also be assessed in a Gli-1/LacZ transgenic mouse model (where the beta-Galatosidase gene has been knocked into the Gli-1 locus resulting in increased beta-Gal production in all cells that have up-regulation in hedgehog pathway activity).
(iii) TAK1−/− Hepatocyte-specific Transgenic mouse model: In case of positive results in one of the above groups, mice with a hepatocyte-specific deletion of TAK1 will be used as an additional fibrosis model for confirmation of IPI-926 effects. At the age of 6 weeks, mice will be treated with IPI-926 at 40 mg/kg, or vehicle for 2 weeks. Treatment groups of Alb-Cre+ TAK1 fl/fl mice will consist of 10 mice. In addition, control groups of 5 Alb-Cre-mice will be treated with IPI-926 or vehicle.

Fibrosis can be evaluated by monitoring one or more of:
(i) Quantitive real-time PCR for fibrogenic genes (Col1a1, Acta2, TIMP-1, Lox)
(ii) Sirius red staining
(iii) SMA Immunohistochemistry (IHC) and/or Western Blot
(iv) In some cases, fibrosis will be confirmed by hydroxyproline assays
(v) Temporal and spatial Gli-1 activity and SHh expression may be analyzed by IHC (methods have been developed by Infinity)
(vi) The activity of the hedgehog pathway in response to liver fibrotic induction and modulation by IPI-926 may also be assessed by qRT-PCR evaluation of the mRNA levels of Gli-1 and other hedgehog pathway responsive genes (Gli-2, PTCH, WIF-1, IGFBP3).

### 3.3 Experimental Outline for Carbontetrachloride-Induced Hepatic Fibrosis

a. 4 groups of mice are injected with CCl4 twice per week for 4 weeks, 10 mice per group.

i. Group 1: Vehicle control;

ii. Group 2: Prophylactic drug treatment: drug treatment 7-10 days prior to beginning of study and then throughout entire study;

iii. Group 3: Drug treatment arm A: drug treatment coinciding with beginning of CCl4 administration and then throughout entire study;

iv. Group 4: Drug treatment arm B: drug treatment coinciding with onset of disease (after 2 weeks' of CCl4 treatment) and then throughout entire study;

b. Mice are terminated 48 hrs after last CCl4 injection. Endpoints:

i. Serum biochemistry: ALT, AST, bilirubin;

ii. Liver histopathology: HE staining and Sirius red staining;

c. Optional endpoints:

i. Fibrosis biomarkers: MMP, collagen I, HGF, TGF-beta by qRT-PC;

ii. Immunohistochemistry for a-SM;

iii. Hepatocyte regeneration: Ki67 by IHC;

iv. Apoptosis: TUNEL.

### 3.4 Experimental Procedures for Carbontetrachloride-Induced Hepatic Fibrosis Animals

Female BALB/c mice (6-7 weeks old; 18-25 g) are used in the present study. Animals are housed at 20-25° C. and 40-70% humidity and subjected to a 12-hour light/dark cycle with access to food and water ad libitum. A total of 73 mice are used in this study. All animals are housed for acclimation for no less than 7 days.

Reagents, references, and test article:

### Olive oil: Sinopharm Chemical, Cat: 69018029.

Carbon tetrachloride (CCl4): China-reagent Co., Ltd, Lot: 20081012.
Test article: IPI-926 will be dosed orally @ 40 mg/kg every other day (QOD).

### Treatment Groups

Following the acclimation period, mice are randomly divided into six groups with a similar average body weight in each group:

i) Group 1: Naïve (n=3);

ii) Group 2: CCl4 only (n=10);

iii) Group 3: CCl4 with Vehicle Group (n=15);

iv) Group 4: CCl4 with Prophylactic IPI-926 treatment (n=15): drug treatment begins 10 days prior to CCl4 treatment and then throughout the entire study;

v) Group 5: CCl4 with IPI-926 treatment arm A (n=15): drug treatment coinciding with the beginning of CCl4 administration and then throughout the entire study;

vi) Group 6: CCl4 with IPI-926 treatment arm B (n=15): drug treatment coinciding with onset of disease (after 2 weeks' of CCl4 treatment) and then throughout entire study;

From Day −10 and then throughout the entire study, animals in Group 4 are given prophylactic drug treatment orally at 40 mg/kg every other day, (PO, QOD). Animals in Group 3 are given Vehicle 5% HPBCD [(2-Hydroxylpropyl)-β-Cyclodextrin] in WFI (Water for Injection) on the same schedule.

From Day 0, all animals except Group 1 are injected intraperitoneally with 1 ml/kg 25% CCl4 in olive oil (100 μl for a regular mouse with body weight of 25 g) twice per week for 4 weeks.

Mice in Group 5 are given drug treatment (40 mg/kg, PO, QOD) coinciding with the beginning of CCl4 administration (at least 30 min after CCl4 injection) and then throughout the entire study. Group 6 mice are given drug treatment (40 mg/kg, PO, QOD) coinciding with the onset of disease (after 2 weeks' CCl4 treatment) and then throughout the entire study.

One day after the last dosage of CCl4, mice are fasted overnight. On the day of sacrifice (e.g. 48 hrs after the last CCl4 injection), 300 μl blood sample per animal is collected under anesthesia with isoflurane. Serum samples will be prepared by centrifugation at 2000×g for 10 min at room temperature. All the serum samples are stored at −80° C. for shipping to a sponsor. After blood sampling, the animals will be terminated with CO₂.

### Isolation of Liver for Histology

Isolated whole livers are quickly flushed with PBS, blotted briefly on paper towel, and weighed. A small piece of liver (˜100 mg) is cut, snap-frozen in liquid nitrogen and stored at −80° C. for future analysis. Two small pieces of liver are cut (˜100 mg each) and snap-frozen in liquid nitrogen for RNA extraction. One separate set of RNA samples will be stored at −80° C. until being shipped to sponsor together with the serum samples. The other set of RNA samples are subjected to qRT-PCR for the following targets: MMP1, collagen 1, hepatocyte growth factor (HGF), TGF-beta.

A small piece of liver is cut, fixed in 10% neutral formalin at 4° C. for 24-48 hrs, and then further processed for paraffin embedding and sectioning. Liver slides are subjected to HE staining, Picro-Sirius red staining and quantitation.

### REFERENCES

The following publications were used in designing the protocols outlined in Example 3:
Roderfeld M, et al Inhibition of hepatic fibrogenesis by matrix metalloproteinase-9 mutants in mice FASEB J. 2006 March; 20(3):444-54; Moreno M, et al. Ghrelin attenuates hepatocellular injury and liver fibrogenesis in rodents and influences fibrosis progression in humans. Hepatology. 2010 March; 51(3):974-85; Novobrantseva T, et al. Attenuated liver fibrosis in the absence of B cells. J Clin Invest. 2005; 115:3072-82; Son G, et al. Selective inactivation of NF-kappaB in the liver using NF-kappaB decoy suppresses CCl4-induced liver injury and fibrosis. Am J Physiol Gastrointest Liver Physiol. 2007 September; 293(3):G631-9; Tong Z, et al. Susceptibility to liver fibrosis in mice expressing a connective tissue growth factor transgene in hepatocytes. Hepatology. 2009 September; 50(3):939-47; Batts K P and Ludwig J. Chronic hepatitis. An update on terminology and reporting. Am J Surg Pathol 1995; 19: 1409-17; Ishak K, et al. Histological grading and staging of chronic hepatitis. J Hepatol 1995; 22: 696-9; and Da Silva Morais A, et al. Peroxisome proliferated-activated receptor gamma ligand, Pioglitazone, does not prevent hepatic fibrosis in mice. Int J Mol. Med. 2007 January; 19(1):105-12.

### Example 4

### Role of Hedgehog Pathway in Primary Myelofibrosis

### 4.1 Background

Primary myelofibrosis (PMF) is a clonal disorder of multipotent hematopoietic progenitor cells. The disease is characterized by anemia, splenomegaly and extramedullary hematopoiesis, and is marked by progressive marrow fibrosis and atypical megakaryocytic hyperplasia. CD34+ stem/progenitor cells abnormally traffic in the peripheral blood and multi organ extramedullary erythropoiesis is a hallmark of the disease, especially in the spleen and liver. The bone marrow structure is altered due to progressive fibrosis, neoangiogenesis, and increased bone deposits. A significant percentage of patients with PMF have gain-of-function mutations in genes that regulate hematopoiesis, including Janus kinase 2 (JAK2) (˜50%) or the thrombopoietin receptor (MPL) (5-10%), resulting in abnormal megakaryocyte growth and differentiation. Studies have suggested that the clonal hematopoietic disorder leads to secondary proliferation of fibroblasts and excessive collagen deposition.

The inventors have demonstrated that hedgehog (Hh) signaling between pancreatic tumor cells and the surrounding stroma plays a role in fibrosis associated with pancreatic cancer. Without wishing to be bound by theory, Hh ligand-dependent activation of the Hh pathway is believed to occur in myelofibrosis, either through ligand expressed by the abnormal stem cells or in the surrounding stroma or both Inhibition of Hedgehog pathway signaling by an inhibitor of Smoothened can result in decreased fibrosis, as seen in nonclinical models of pancreatic cancer. Decreased bone marrow fibrosis can improve clinical signs and symptoms, including anemia, abnormal leukocyte counts, and splenomegaly.

### 4.2 Non-Clinical Experiments

Hedgehog Pathway expression can be evaluated in bone marrow biopsies taken from patients with myelofibrosis, and then compared to normal bone marrow biopsy tissue using:

1) Immunohistochemistry for Hh ligand (Shh, Ihh), Gli-1, primary cilia, and/or

2) RT-PCR for SHh, IHh, DHh, Smo, Gli-1, Gli-2, Ptch-1, WIF-1,

### 4.3 Clinical Experiments

The activity of IPI-926 for the treatment of myelofibrosis, either as a single and/or in combination with other agents, can be evaluated in clinical trials. Combination treatments can involve IPI-926 in combination with agents such as e.g., Jak2 inhibitors (including, but not limited to INCB018424, XL019, and TG101348, TG101209); immunomodulators such as IMIDs (including, but not limited to thalidomide, lenalidomide, panolinomide); hydroxyurea; androgens; prednisone; danazol, and other agents. These trials can evaluate whether Hh pathway inhibition with a Smo inhibitor will decrease fibrosis and improve blood counts in patients with PMF. Following daily oral administration of IPI-926, bone marrow samples will be evaluated every three months for evidence of Hh pathway inhibition and decreased fibrosis. Initial clinical studies will enroll a small number of patients (10-20), then expand to a larger number if favorable responses to IPI-926 are observed.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A method for reducing fibrosis in a cell or tissue, by contacting the cell or tissue with a hedgehog inhibitor in an amount sufficient to decrease or inhibit the fibrosis.

2. A method of treating a fibrotic condition comprising administering a hedgehog inhibitor to a subject in need thereof, in an amount sufficient to decrease or inhibit the fibrotic condition in the subject.

3. The method of claim 2, wherein treating the fibrotic condition comprises reducing or inhibiting one or more of: formation or deposition of tissue fibrosis; or reducing the size, cellularity, composition, cellular or collagen content, of a fibrotic lesion.

4. The method of claim 2, wherein the fibrotic condition is a fibrotic condition of the lung, a fibrotic condition of the liver, a fibrotic condition of the heart or vasculature, a fibrotic condition of the kidney, a fibrotic condition of the skin, a fibrotic condition of the gastrointestinal tract, a fibrotic condition of the bone marrow or hematopoietic tissue, a fibrotic condition of the nervous system, or a combination thereof.

5. The method of claim 2, wherein the fibrotic condition is secondary to an infectious disease, an inflammatory disease, an autoimmune disease, a connective disease, a malignant disorder or a clonal proliferative disorder; a toxin; an environmental hazard, cigarette smoking, a wound; or a medical treatment chosen from a surgical incision, chemotherapy or radiation.

6. The method according to claim 2, wherein the fibrotic condition a fibrotic condition of the lung.

7. The method according to claim 6, wherein the fibrotic condition of the lung is chosen from one or more of: pulmonary fibrosis, idiopathic pulmonary fibrosis (IPF), usual interstitial pneumonitis (UIP), interstitial lung disease, cryptogenic fibrosing alveolitis (CFA), or bronchiectasis.

8. The method according to claim 2, wherein the fibrotic condition is a fibrotic condition of the liver.

9. The method according to claim 8, wherein the fibrotic condition of the liver is chosen from fatty liver disease, steatosis, primary biliary cirrhosis (PBC), cirrhosis, alcohol induced liver fibrosis, biliary duct injury, biliary fibrosis, hepatic fibrosis associated with hepatitis infection, autoimmune hepatitis, non-alcoholic fatty liver disease (NAFLD), or progressive massive fibrosis.

10. The method according to claim 2, wherein the fibrotic condition is a fibrotic condition of the heart or vasculature.

11. The method according to claim 10, wherein the fibrotic condition of the heart or vasculature is myocardial fibrosis.

12. The method according to claim 2, wherein the fibrotic condition is a fibrotic condition of the kidney.

13. The method according to claim 12, wherein the fibrotic condition of the kidney is chronic kidney fibrosis, nephropathies associated with injury/fibrosis, diabetic nephropathy, lupus, scleroderma of the kidney, glomerular nephritis, focal segmental glomerular sclerosis, IgA nephropathyrenal fibrosis associated with human chronic kidney disease (CKD), chronic progressive nephropathy (CPN), tubulointerstitial fibrosis, ureteral obstruction, chronic uremia, chronic interstitial nephritis, radiation nephropathy, glomerulosclerosis, progressive glomerulonephrosis (PGN), endothelial/thrombotic microangiopathy injury, or HIV-associated nephropathy.

14. The method according to claim 2, wherein the fibrotic condition is a fibrotic condition of the skin.

15. The method according to claim 14, wherein the fibrotic condition of the skin is selected from skin fibrosis, scleroderma, nephrogenic systemic fibrosis, and keloid.

16. The method according to claim 2, wherein the fibrotic condition is a fibrotic condition of the gastrointestinal tract.

17. The method according to claim 16, wherein the fibrotic condition of the gastrointestinal tract is diffuse scleroderma of the gastrointestinal tract.

18. The method according to claim 2, wherein the fibrotic condition is a fibrotic condition of the bone marrow.

19. The method according to claim 2, wherein the fibrotic condition of the bone marrow or hematopoietic tissue is chosen from one or more of: primary myelofibrosis; a fibrosis associated with a hematologic disorder chosen from polycythemia vera, essential thrombocythemia, myelodysplasia, hairy cell leukemia, lymphoma or multiple myeloma; a fibrosis of secondary to a non-hematologic disorder chosen from solid tumor metastasis to the bone marrow, an autoimmune disorder; an infection; or secondary hyperparathyroidism.

20. The method according to claim 2, wherein the hedgehog inhibitor is the compound of formula (I):
or a pharmaceutically acceptable salt and/or solvate thereof;
wherein:
R¹ is H, alkyl, —OR, amino, sulfonamido, sulfamido, —OC(O)R⁵, —N(R⁵)C(O)R⁵, or a sugar;
R² is H, alkyl, alkenyl, alkynyl, aryl, cycloalkyl, nitrile, or heterocycloalkyl;
or R¹ and R² taken together form ═O, ═S, ═N(OR), ═N(R), ═N(NR₂), or ═C(R)₂;
R³ is H, alkyl, alkenyl, or alkynyl;
R⁴ is H, alkyl, alkenyl, alkynyl, aryl, cycloalkyl, heterocycloalkyl, aralkyl, heteroaryl, heteroaralkyl, haloalkyl, —OR, —C(O)R⁵, —CO₂R⁵, —SO₂R⁵, —C(O)N(R⁵)(R⁵), —[C(R)₂]_{q}—R⁵, —[(W)—N(R)C(O)]_{q}R⁵, —[(W)—C(O)]_{q}R⁵, —[(W)—C(O)O]_{q}R⁵, —[(W)—OC(O)]_{q}R⁵, —[(W)—SO₂]_{q}R⁵, —[(W)—N(R⁵)SO₂]_{q}R⁵, —[(W)—C(O)N(R⁵)]_{q}R⁵, —[(W)—O]_{q}R⁵, —[(W)—N(R)]_{q}R⁵, —W—NR₃⁺X⁻ or —[(W)—S]_{q}R⁵; wherein each W is independently for each occurrence a diradical; each q is independently for each occurrence 1, 2, 3, 4, 5, or 6; and X⁻ is a halide;
each R⁵ is independently for each occurrence H, alkyl, alkenyl, alkynyl, aryl, cycloalkyl, heterocycloalkyl, aralkyl, heteroaryl, heteroaralkyl or —[C(R)₂]ₚ—R⁶; wherein p is 0-6; or any two occurrences of R⁵ on the same substituent can be taken together to form a 4-8 membered optionally substituted ring which contains 0-3 heteroatoms selected from N, O, S, and P; and
each R⁶ is independently hydroxyl, —N(R)COR, —N(R)C(O)OR, —N(R)SO₂(R), —C(O)N(R)₂, —OC(O)N(R)(R), —SO₂N(R)(R), —N(R)(R), —COOR, —C(O)N(OH)(R), —OS(O)₂OR, —S(O)₂OR, —OP(O)(OR)(OR), —NP(O)(OR)(OR), or —P(O)(OR)(OR);
each R is independently H, alkyl, alkenyl, alkynyl, aryl, cycloalkyl or aralkyl;
provided that when R², R³ are H and R⁴ is hydroxyl; R¹ cannot be hydroxyl;
provided that when R², R³, and R⁴ are H; R¹ cannot be hydroxyl; and
provided that when R², R³, and R⁴ are H; R¹ cannot be sugar.

21. The method according to claim 20, wherein the compound of formula (I) is a compound provided in Table 1, or a pharmaceutically acceptable salt and/or solvate thereof.

22. The method according to claim 20, wherein the compound of formula (I) is a compound of formula 32:
or a pharmaceutically acceptable salt and/or solvate thereof.

23. The method according to claim 2, wherein the hedgehog inhibitor is administered as a single agent or in combination with a second agent.

24. The method according to claim 23, wherein the fibrotic condition of the bone marrow is treated, and the second agent is chosen from a Jak2 inhibitor, an IMID, hydroxyurea, an androgen, prednisone, or danazol.

25. The method according to claim 2, further comprising the step of monitoring the subject for a change in one or more of: fibrotic lesion size; cellularity, composition, immune/inflammatory levels, hedgehog level or signaling; or stromal activation.

26. The method according to claim 2, further comprising the step of detecting elevated hedgehog ligand in the subject, prior to, or after, administering a hedgehog inhibitor to the patient.

27. A composition for use in reducing fibrosis, or treating a fibrotic condition comprising a hedgehog inhibitor.
